(19) United States
(12) Patent Application Publication
(10) Pub. No.: US 2025/0359987 A1
(43) Pub. Date: Nov. 27, 2025

(54) ARTIFICIAL HEART VALVE, DEVICE FOR PERFORMING A CATHETER-BASED IMPLANTATION OF AN ARTIFICIAL HEART VALVE, DEVICE FOR AN IN-SITU REPLACEMENT OF AN INNER ARTIFICIAL HEART VALVE, METHOD FOR EXPLANTING AN ARTIFICIAL HEART VALVE, AND METHOD FOR REIMPLANTING AN ARTIFICIAL HEART VALVE

(71) Applicant: CORMOS MEDICAL GMBH, Jena (DE)

(72) Inventor: Robert Moszner, Bad Klosterlausnitz (DE)

(21) Appl. No.: 18/865,455

(22) PCT Filed: May 12, 2023
(86) PCT No.: PCT/EP2023/062697

§ 371 Date: Dec. 3, 2024

(30) Foreign Application Priority Data
May 13, 2022 (DE) 10 2022 001 751.2
May 10, 2023 (DE) 10 2023 112 240.1

(51) Int. Cl. A61F 2/24

(52) U.S. Cl. CPC A61F 2/2418; A61F 2/2436; A61F 2210/0014; A61F 2220/0025; A61F 2250/0069; A61F 2250/0098

(57) ABSTRACT

The invention relates to an artificial heart valve having a two-part design, consisting of an outer artificial heart valve having means for permanently anchoring the artificial heart valve at the implantation site and an inner artificial heart valve, which can be replaced independently of the outer artificial heart valve anchored at the implantation site and can be inserted into the outer artificial heart valve and which has artificial heart valve cusps, wherein the outer artificial heart valve has formations for defined placement of the inner artificial heart valve and the inner artificial heart valve for anchoring the inner artificial heart valve in the outer artificial heart valve. The invention also relates to a device for performing a catheter-based implantation of an artificial heart valve, to a method for explanting an artificial heart valve, and to a method for reimplanting an artificial heart valve.

### DESCRIPTION

The invention relates to an interventional replaceable two-piece artificial heart valve according to claim 1, a device for performing a catheter-based implantation of an artificial heart valve according to claim 8, a device for an in-situ replacement of an inner artificial heart valve according to claim 10, a method for explanting an artificial heart valve according to claim 15 and a method for reimplanting an artificial heart valve according to claim 18.

The human heart contains four heart valves whose function ensures a directed blood flow. These are the aortic valve as part of the ejection tract of the left heart in the transition to the aorta of the large circulation, the pulmonary valve as part of the ejection tract of the right heart in the transition to the pulmonary artery of the small circulation, the tricuspid valve between the right atrium and the right ventricle and the mitral valve between the left atrium and the left ventricle.

All of the valves mentioned can lose functionality in connection with age-related heart changes or heart disease. These include, in particular, valve stenosis, in which the valves do not open properly and therefore no longer form a physiological resistance to the blood flow, insufficiency, in which the closing function of the valves is impaired, resulting in a non-physiological backflow of blood, but also combinations of stenosis and insufficiency, in which the valve in question neither opens nor closes sufficiently.

The aortic valve, for example, is one of the four heart valves in the human heart. In terms of its anatomical structure and physiological function, it belongs to the so-called pocket valves. The aortic valve is located between the left ventricular outflow tract, i.e. the left ventricle, and the adjoining ascending aorta. It prevents diastolic backflow of blood from the aorta into the left ventricle. The aortic valve is usually made up of three pockets, although a small proportion of the population may have two or four pockets.

Diseases of the heart valves, particularly the aortic valve, are different for each valve in clinical practice, but are relatively common overall and are particularly prevalent in older patients. Mechanical and biological valve prostheses are available for the treatment of diseased and functionally impaired heart valves, which are used surgically or interventional via a catheter-based procedure. A catheter is inserted into the patient's aortic arch via an arterial access, through which the valve prosthesis is inserted into the area of the aortic valve.

Heart valve prostheses are used to treat heart valve stenosis. An interventional catheter-based heart valve implantation procedure (Transcatheter Aortic Valve Implantation—TAVI) has become established as a treatment method. A major advantage of this catheter-based implantation procedure is that no extracorporeal membrane oxygenation (ECMO) is required to implant the heart valve prosthesis. ECMO is a support system used in intensive care medicine in which a machine takes over part or all of the respiratory function for the patient outside the body. Such systems are also known as “heart-lung machines”.

The example given here with regard to the aortic valve also applies analogously to the other heart valves of the human heart. All heart valves can be treated in the same way as the TAVI implantation procedure, although the delivery routes of the corresponding catheter are of course different.

Surgical valve replacement as a device for the treatment of heart valve diseases is also known from medical technology. Both mechanical and biological prostheses are available for this purpose. As a rule, these artificial heart valves are surgically sutured into place. The chest must be opened for this. After removing the degenerated native heart valve, the surgical valve replacement is sutured into the patient. During the procedure, the patient must be connected to a heart-lung machine (ECMO) and cardiac arrest must be induced when the valve replacement is implanted. The surgical procedure is associated with a long post-operative treatment phase and is very stressful for the patient. Particularly in older patients, such an operation can no longer be performed with an acceptable level of risk.

Interventional valve replacement using the technical principle of transcatheter artificial heart valve is a gentler procedure compared to surgical therapy. In this procedure, a self-expanding stent combined with folded biological heart valves is implanted in a corresponding sheath system in the patient. At the implantation site, the stent, which is composed of several self-expanding molded elements that can be angled towards each other in its longitudinal alignment, can be deployed step by step according to axial alignment by means of an inflatable balloon mechanism or an equivalent expansion mechanism. As it unfolds, the diseased native heart valve is pushed aside by the artificial valve replacement.

Despite the progressive development of interventional valve replacement technology, problems still occur. This particularly concerns the anchoring and positioning of the prostheses. During the filling phase of the ventricles, considerable forces are exerted on the valve. To counteract these forces, the valve prosthesis must be securely anchored to prevent the implanted medical device from detaching and the associated axial migration. Some current products on the market have insufficient radial support force, are difficult to attach and are too long from the main body. These problems occur particularly with aortic valve prostheses. This promotes atrioventricular block (i.e. a certain type of cardiac arrhythmia), postoperative paravalvular leakage, strokes or organ fatigue. Furthermore, incorrect implantations occur time and again, for example if the prosthesis is not optimally positioned or in the case of anatomical anomalies. These can lead to paravalvular leakages or blockages of the adjacent coronary arteries, among other things, which can have considerable consequences for the patient.

Other problems relate to the materials of the heart valves. Biological heart valves are commonly used.

Biological valves are devices that are preferably obtained from processed animal tissue and used for implantation in humans. The tissue is either heart valve leaflets, for example from pigs, the so-called porcine valve, or valve materials made from the pericardium of cattle. The latter valves are known as bovine valves.

The main component of biological tissue is collagen, which, however, would rapidly degrade after implantation in humans if left untreated. For clinical application, tissue cross-linking is therefore necessary, which can be achieved by fixation with chemical detergents. One standardized method is the use of aldehydes, including glutaraldehyde. The aldehyde groups cross-link the primary amino groups of the collagen molecules in the tissue and thus increase the resistance of the tissue, including reduced immunization strength and increased stability. Despite the widespread use of tissue fixation, aldehydes, but also other chemical cross-linking agents, show an increased susceptibility to the development of degenerative calcifications after implantation. A so-called pathological calcification (calcinosis), triggered by the binding of free calcium salts to free aldehyde groups and exposed acidic phospholipids, leads to stiffening of the valve and thus to loss of physiological function.

Treatment of fixed tissue with non-covalent binding detergents, such as sodium dodecyl sulphate (SDS), Tween-80 (a polysorbent) or lower alcohols (e.g. ethanol) to remove phospholipids, or covalent binding detergents, such as AOA or L-glutamic acid to bind free aldehyde groups, significantly reduce the calcification of materials exposed to circulating blood. However, the effect of detergents is only temporary. The detergents can also have a negative effect on the tissue structure and/or tissue properties, in particular strength and durability. Despite the treatment, the degeneration of the biological prosthesis cannot be completely prevented, it is merely delayed.

Replacement of the biological valve is therefore necessary on average after 10-15 years due to the progressive calcification of the valve, combined with increasing valve insufficiency. According to current prior art, valve replacement can only be carried out surgically by opening the chest, which represents an increased risk, especially for older patients. Therefore, according to current guidelines, the use of biological valves is primarily recommended for older patients. In young patients, the tendency is to use mechanical prostheses.

In catheter-based implantations, an artificial valve is placed interventional on the defective native heart valve. This procedure is used in particular for the aortic valve. The prostheses used for this type of implantation procedure basically consist of a metallic frame, which in turn serves to fix several integrated valve leaflets made of a biological material. For the biological material, for example, materials from cattle or pigs are used.

However, the functional life of such biological heart valve prostheses is limited as described. The limited functional life is primarily caused by natural degeneration, in particular calcification, of the prosthesis, which usually occurs after 10 to 15 years. The heart valve prosthesis then loses its function. After the loss of function has occurred, such conventional biological heart valve prostheses must be replaced by open heart surgery according to the current prior art.

To date, all new heart valves have been implanted surgically, minimally invasively and interventional. However, these implanted heart valve prostheses lose quality and function to varying degrees over time. Implanted heart valves, for example, lose their function after approx. 10-15 years due to the degeneration of the biological valve leaflets. The heart valve prosthesis must therefore be replaced in good time. One option is to replace the heart valve prosthesis by means of open heart surgery or catheter-based (interventional) surgery. Recently, interventional implantations have been performed more and more frequently; currently, this procedure already accounts for two-thirds of all interventions and is growing rapidly.

The removal and replacement of defective heart valves in particular is currently always carried out using open heart surgery. This operation is considerably more risky for the patient than an interventional procedure using the much less risky catheter technique. However, it is currently unavoidable.

The object is therefore to remedy the described disadvantages existing in the prior art. In particular, the object is to enable an easy-to-handle interventional Implantation procedure with safe implantation completion and to create a corresponding artificial heart valve for this purpose. In addition, the dimensions of the catheters used are also to be reduced.

The solution to said object is achieved with an artificial heart valve according to claim 1, a device for performing a catheter-based implantation of an artificial heart valve according to claim 8, a device for an in-situ replacement of an inner artificial heart valve according to claim 10, a method for explanting an artificial heart valve according to claim 15 and a method for reimplanting an artificial heart valve according to claim 18. The corresponding subclaims contain useful and advantageous embodiments of the respective device or method.

The artificial heart valve according to the invention has a two-piece structure, consisting of an outer artificial heart valve with means for permanently anchoring the artificial heart valve at the implantation site and an inner artificial heart valve with artificial heart valve pockets which can be replaced independently of the outer artificial heart valve anchored at the implantation site and inserted into the outer artificial heart valve, wherein the outer artificial heart valve has formations for a defined positioning of the inner artificial heart valve and the inner artificial heart valve has formations for anchoring the inner artificial heart valve in the outer artificial heart valve.

In an advantageous design, the outer artificial heart valve is an arrangement of closed molded elements cut from a tube in the shape of a net, wherein the formations for the defined support are a series of shoulders projecting into the interior of the arrangement. The inner artificial heart valve has a shape-variably compressible wire-like meandering structure of open molded elements, wherein the formations for anchoring are designed as a series of first anchors and second anchors and the inner artificial heart valve has a series of segments for fastening the artificial heart valve pockets.

In a further optional addition, the outer artificial heart valve has a patch sheath surrounding its distal end to seal paravalvular leaks.

In an advantageous design, the inner artificial heart valve and the outer artificial heart valve consist of a rotationally symmetrical, elastic nitinol sheath, wherein the open molded elements and the closed molded elements are cut out of the nitinol sheath.

In addition, the outer artificial heart valve in the region of the protruding shoulders and the inner artificial heart valve in the region of the first anchors and/or the second anchors can each have sections forming X-ray contrasts as markers.

A microprocessor can be arranged in the region of each marker. The microprocessor is a read-only memory with a unique identifier that can be read out with an approaching readout means. This allows axial or azimuthal orientations to be automatically detected and subsequently converted into control pulses for a corresponding automatically effective adaptive positioning mechanism in conjunction with an implantation tool.

The inner artificial heart valve and/or the outer artificial heart valve can have a non-stick coating and/or a pharmacologically effective coating. This can prevent the formation of thrombi in particular.

The device according to the invention for performing a catheter-based implantation of an artificial heart valve consists of the following components:

According to the invention, there is a tube-like flexible implantation sheath, a guide wire movable within the initial implantation sheath and the two-piece artificial heart valve, which is displaceable within the initial implantation sheath along the guide wire and consists of an outer artificial heart valve and an inner artificial heart valve or a displaceable inner heart valve insert for insertion into an already pre-implanted outer heart valve insert.

In addition, the initial implantation sheath can be supplemented by a tubular-flexible catch net sheath having a particle catch net that can be unfolded at the distal end and covers the valve cross-section, wherein the initial implantation sheath can be passed through the particle catch net from the outside to the intended implantation site. The particle catch net is used to trap small thrombi, tissue fragments, calcifications and similar components present in the area of the surgical site. This prevents the particles from being carried into the subsequent bloodstream.

The device according to the invention for an in-situ replacement of an inner artificial heart valve with a new inner artificial heart valve in an implanted two-piece artificial heart valve, comprising the inner artificial heart valve and the outer artificial heart valve, consists of the following components:

According to the invention, there is an explantation catheter for an explantation of the inner artificial heart valve, comprising a tubular-flexible explantation sheath with a guide wire arranged in the explantation catheter and an explantation tool guided in the explantation sheath with means for gripping and releasing anchors of the inner artificial heart valve with a proximal operating unit for semi-automatic operation of the explantation tool and an implantation catheter, comprising a tubular-flexible implant exchange sheath with means for feeding and inserting a new inner artificial heart valve into the outer artificial heart valve.

In an advantageous embodiment, the explantation tool has the following structure: A first feed means is provided having a bell-shaped tool shield attached to the distal end of the first feed means and a guide means accommodating the tool shield for guiding the first feed means. There is also provided a second feed means with a continuous recess accommodating the first feed means, as well as a bearing connecting the second feed means to the guide means for the tool shield and fixing the second feed means in the axial direction, and a catch tube tool for the tool shield which can be moved by the second feed means.

In an advantageous design, the first feed means is a first inner screw wire with a tool shield attached to the distal end of the first screw wire and a tool shield threaded sleeve accommodating the tool shield for guiding the first screw wire.

In a further advantageous design, the second feed means is a second outer screw wire with a continuous core bore accommodating the inner screw wire and an outer threaded section with a bearing connecting the second outer screw wire to the threaded sleeve and fixing the outer screw wire in the axial direction. A catch tube threaded sleeve guiding the outer threaded section of the outer screw wire and a catch tube tool connected to the catch tube threaded sleeve and concentrically surrounding the tool shield threaded sleeve are provided.

In a further embodiment, the tool shield is designed in such a way that, in the unfolded state, it completely peripherally covers the inner artificial heart valve located in the outer artificial heart valve up to shoulders located in the outer artificial heart valve and engages in the peripheral space between the inner artificial heart valve and the outer artificial heart valve, wherein anchoring formations of the inner artificial heart valve are released from the outer artificial heart valve by this Intervention and instead engage in perforations of the tool shield.

The method according to the invention for explanting an artificial heart valve comprises the following method steps:

A two-piece artificial heart valve implanted at the implantation site with an outer artificial heart valve firmly implanted at the implantation site and an inner artificial heart valve detachably anchored in the outer artificial heart valve is used.

An explantation catheter with a tubular-flexible explantation sheath and a gripping and removal tool contained in the explantation sheath is then advanced into the region of the implantation site.

The gripping and removal tool is then extended from the explantation sheath onto the inner artificial heart valve.

The inner artificial heart valve is then gripped and the anchors with the outer artificial heart valve are released by the gripping and removal tool.

The gripped inner artificial heart valve is then withdrawn by the grasping and removal tool and the inner artificial heart valve is retrieved in the explantation sheath of the explantation catheter.

Finally, the explantation catheter is withdrawn with the inner artificial heart valve retrieved in the explantation sheath.

In an advantageous design of the method, the gripping and removal tool is extended from the explantation sheath and the inner artificial heart valve is gripped by extending and deploying a tool shield, wherein the tool shield is lowered over the inner artificial heart valve, thereby releasing the anchoring of the inner artificial heart valve to the outer heart valve projection and hooking the inner heart valve projection onto the tool shield.

In an advantageous design of the method, the gripping and removal tool retracts the captured inner artificial heart valve and the captured inner artificial heart valve is retrieved with the following method steps:

The tool shield with the hooked inner artificial heart valve is retracted into a catch tube tool emerging from the explantation sheath and the inner artificial heart valve is compressed in the catch tube tool. The capture tube tool is then retracted into the explantation sheath with the compressed inner artificial heart valve inside.

The method according to the invention for reimplanting an artificial heart valve comprises the following steps:

A two-piece artificial heart valve is used, consisting of an inner artificial heart valve and an outer artificial heart valve, wherein the outer artificial heart valve is firmly implanted in the anatomical region of the heart valve and remains permanently at the implantation site and the inner artificial heart valve is advanced into the region of the pre-implanted outer artificial heart valve by means of an implantation catheter and inserted into the outer artificial heart valve by means of an implantation tool located in the implantation catheter and is anchored via anchoring means located on the inner artificial heart valve.

Advantageously, the explantation and reimplantation of the artificial heart valve is carried out as a directly successive explantation and reimplantation, wherein, in a first step, a first inner artificial heart valve is explanted from the outer artificial heart valve via the explantation catheter and the explantation sheath and, in an immediately subsequent second step, a second inner artificial heart valve is inserted into the outer artificial heart valve via the implantation catheter.

The individual steps during explantation and/or reimplantation are advantageously carried out automatically by means of an automatically operated catheter tool arranged at the proximal end of the explantation catheter and/or the implantation catheter.

Preferred exemplary embodiments of the devices and methods are explained in more detail below. As an example, an artificial heart valve is explained below, which is positioned in the anatomical region of the ejection tract of the left ventricle, i.e. in the transition to the aorta, as an aortic valve replacement. In the case of the aortic valve, the catheter access is obviously femoral arterial.

For the other heart valves, access is adapted according to the anatomical position. Catheter access for the pulmonary and tricuspid valves, for example, is femoral venous and access to the mitral valve is femoral arterial or by means of a puncture via the apex of the heart, i.e. transapical.

The features mentioned in the claims and in the description can each be essential to the invention individually or in any combination. The attached figures serve to illustrate this.

The drawings show as follows:

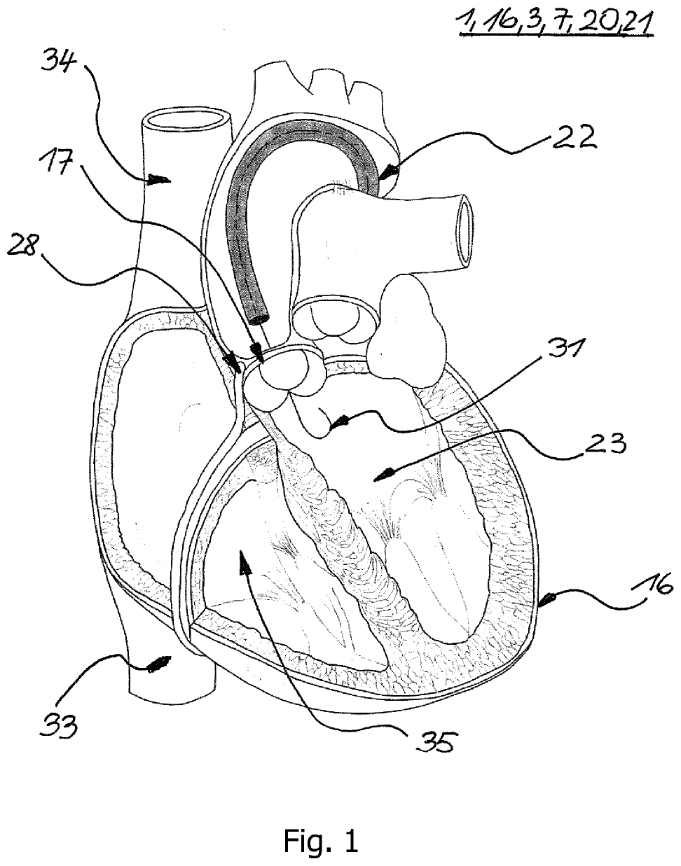

FIG. 1 shows a schematic representation of the side view in conjunction with a partial section of the heart with initial implantation sheath for the implantation procedure;

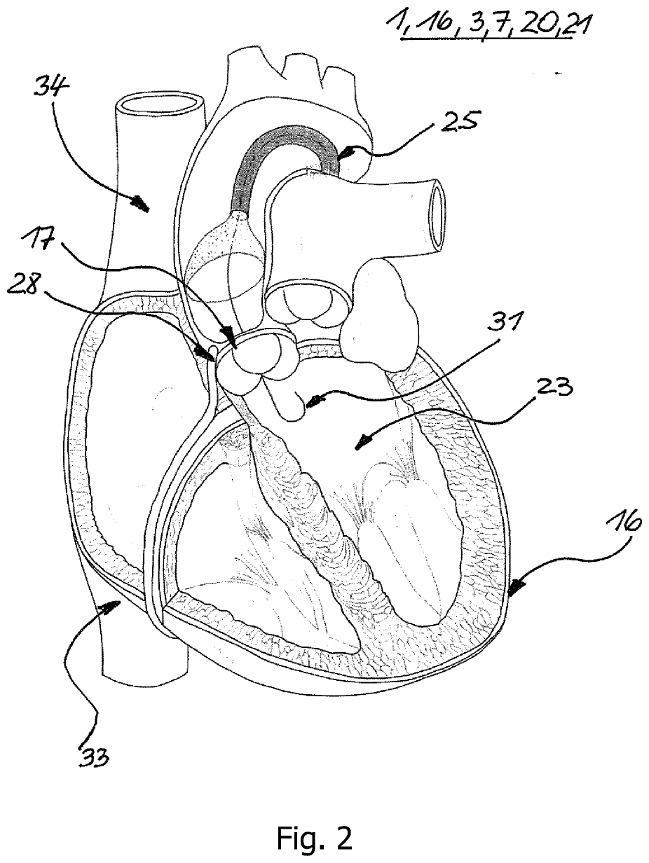

FIG. 2 shows a schematic representation of the side view in conjunction with a partial section of the heart with catch net sheath in conjunction with a catch net;

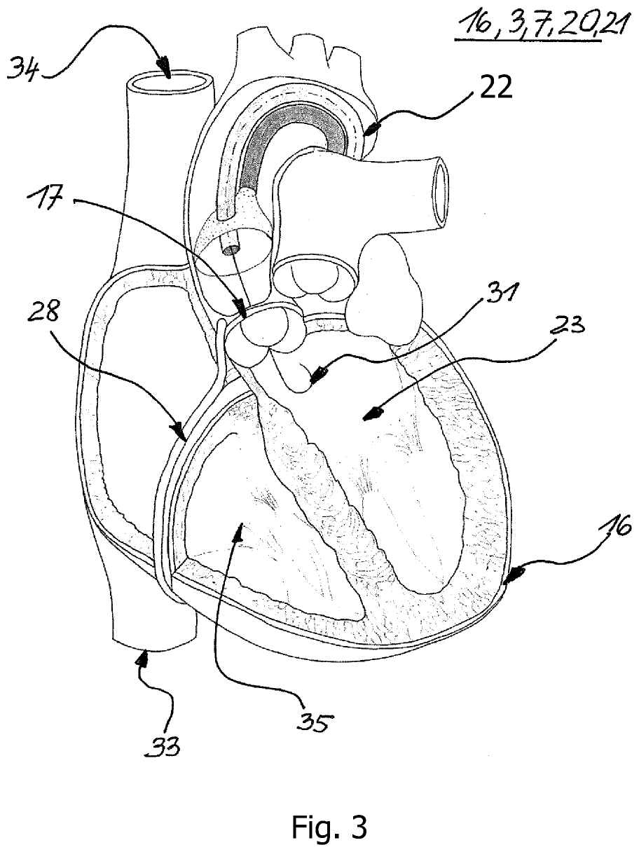

FIG. 3 shows a schematic representation of the side view in conjunction with a partial section of the heart with the initial implantation sheath and the catch net sheath;

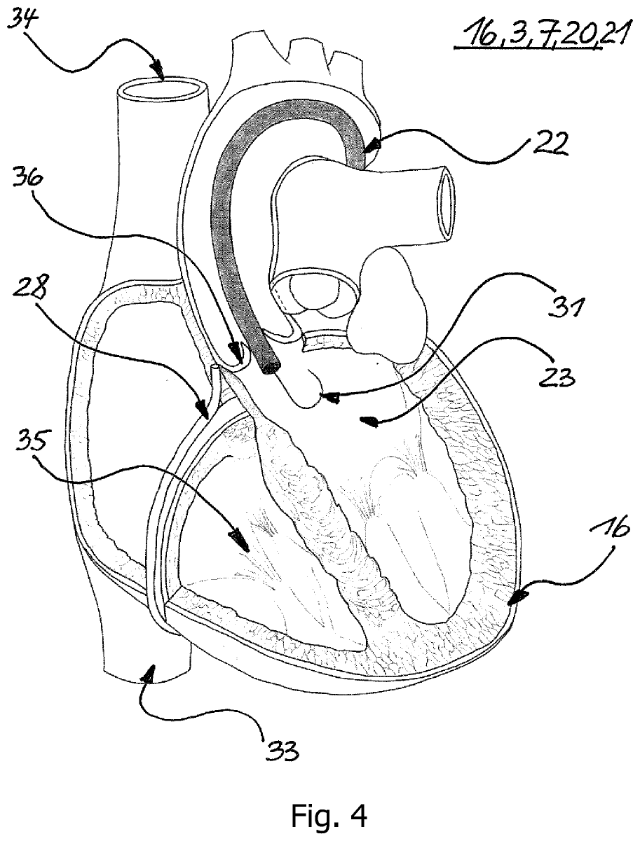

FIG. 4 shows a schematic representation of the side view in conjunction with a partial section of the heart at the beginning of the interventional implantation procedure with initial implantation sheath in accordance with the current TAVI procedure;

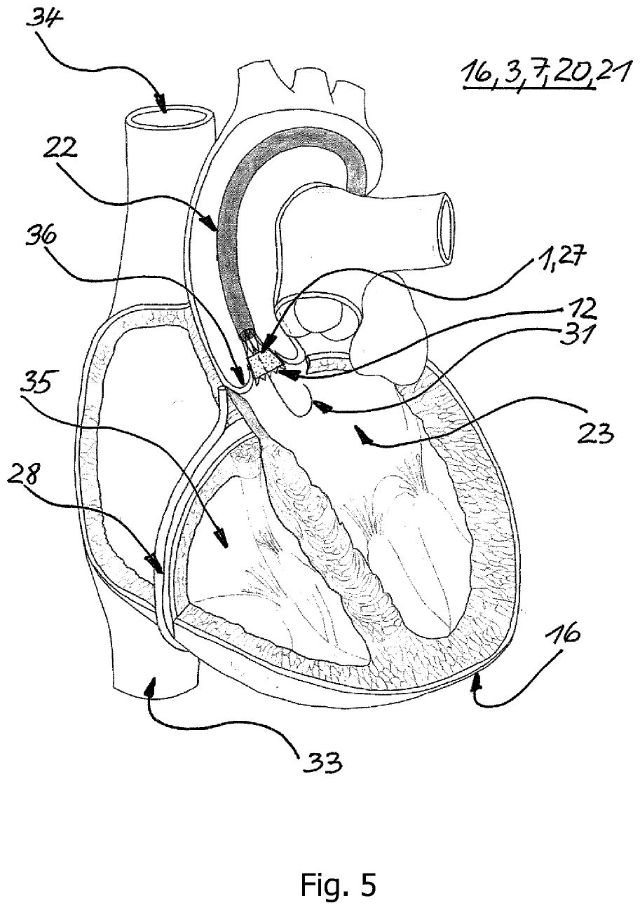

FIG. 5 shows a schematic representation of the side view in conjunction with a partial section of the heart during the further course of the implantation procedure with subsequent release of the two-piece artificial heart valve;

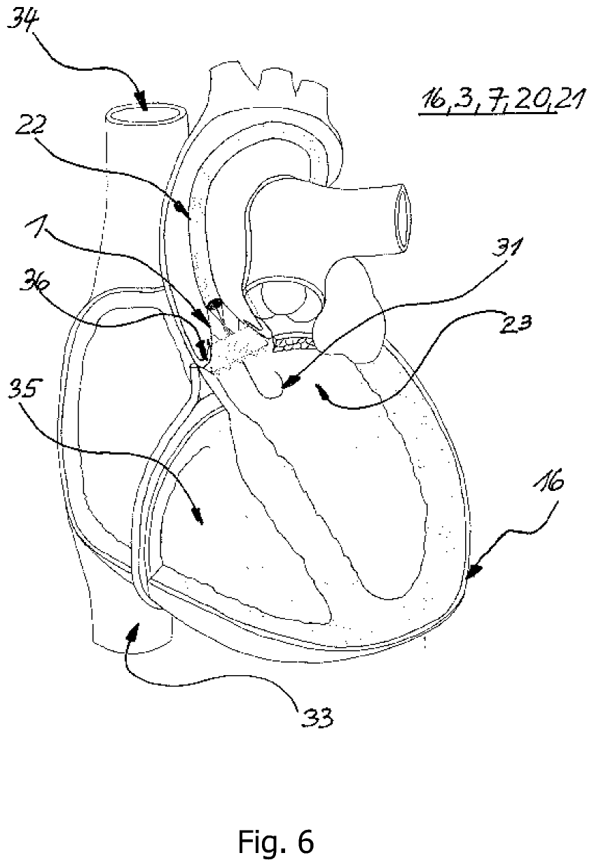

FIG. 6 shows a schematic representation of the side view in conjunction with a partial section of the heart in the advanced course of the implantation procedure with later release of the two-piece artificial heart valve;

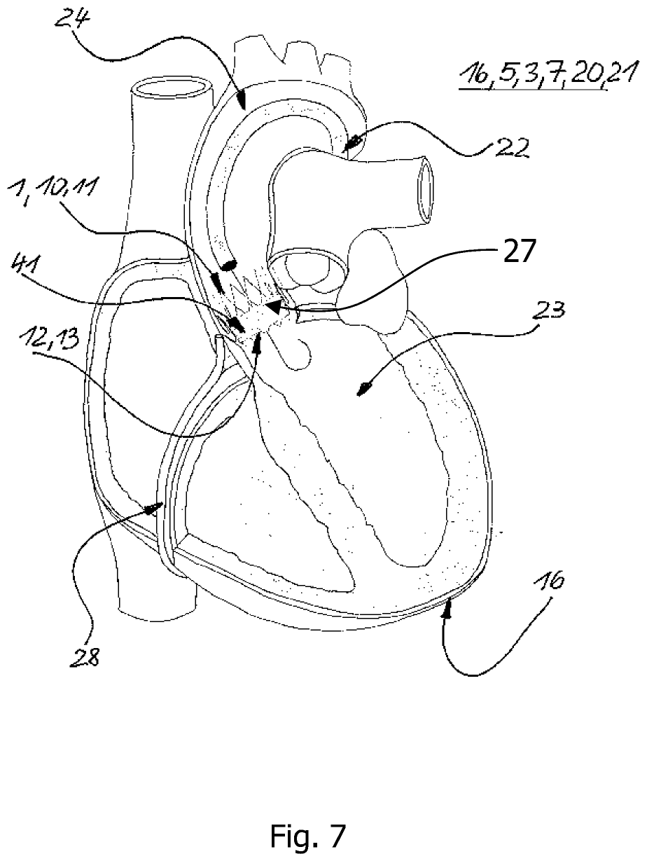

FIG. 7 shows a schematic representation of a detailed side view in conjunction with a partial section of the heart after release of the two-piece artificial heart valve to be implanted;

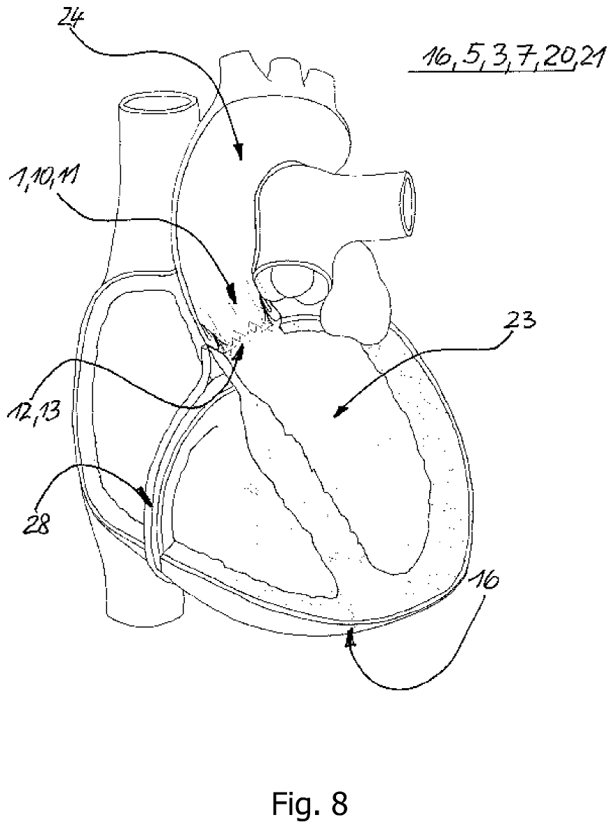

FIG. 8 shows a schematic representation of a detailed side view in conjunction with a partial section of the heart after completion of the implantation procedure;

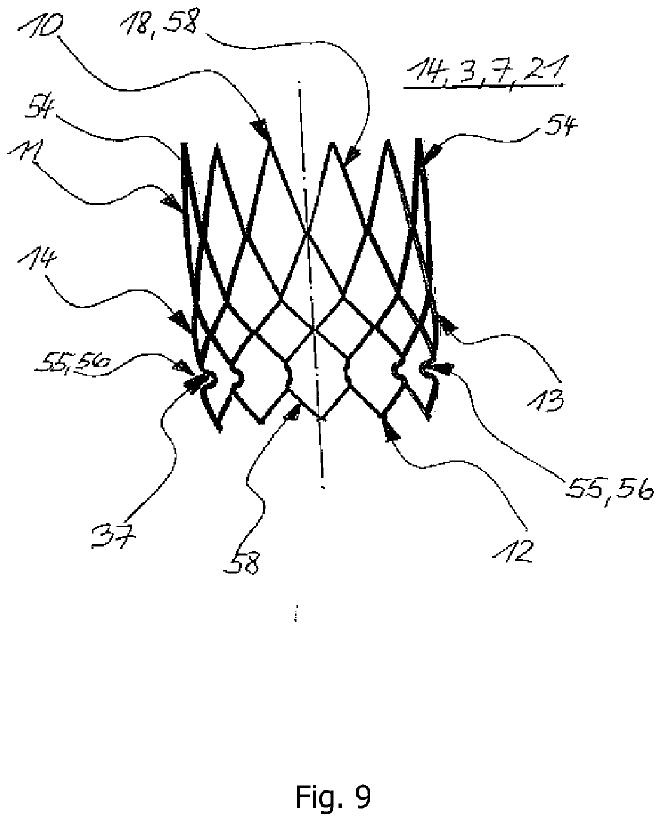

FIG. 9 shows a schematic structure of the outer artificial heart valve as a side view with partial section and predominantly parallelogram-shaped molded elements;

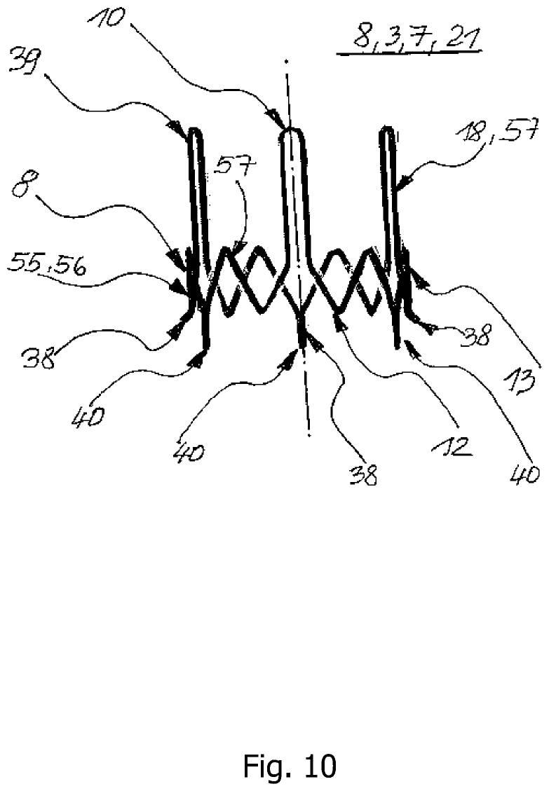

FIG. 10 shows a schematic structure of the inner artificial heart valve with predominantly serrated molded elements, intersecting and not connected to each other;

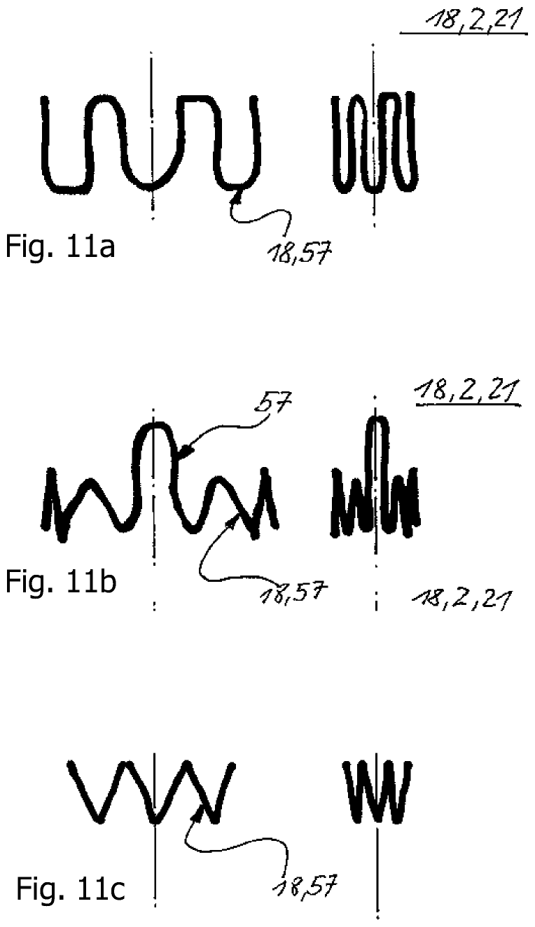

FIG. 11(a-c) shows a schematic representation of molded elements of the inner heart valve;

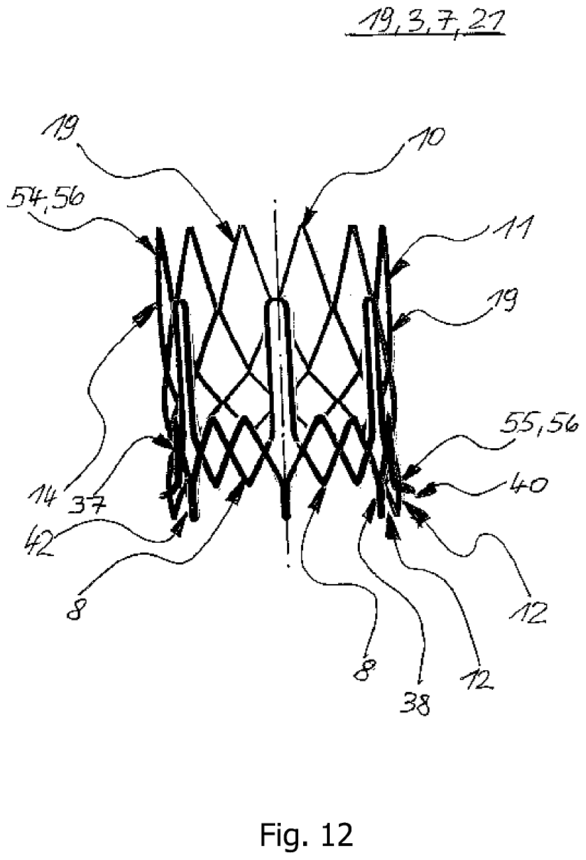

FIG. 12 shows a schematic representation of the two-piece artificial heart valve;

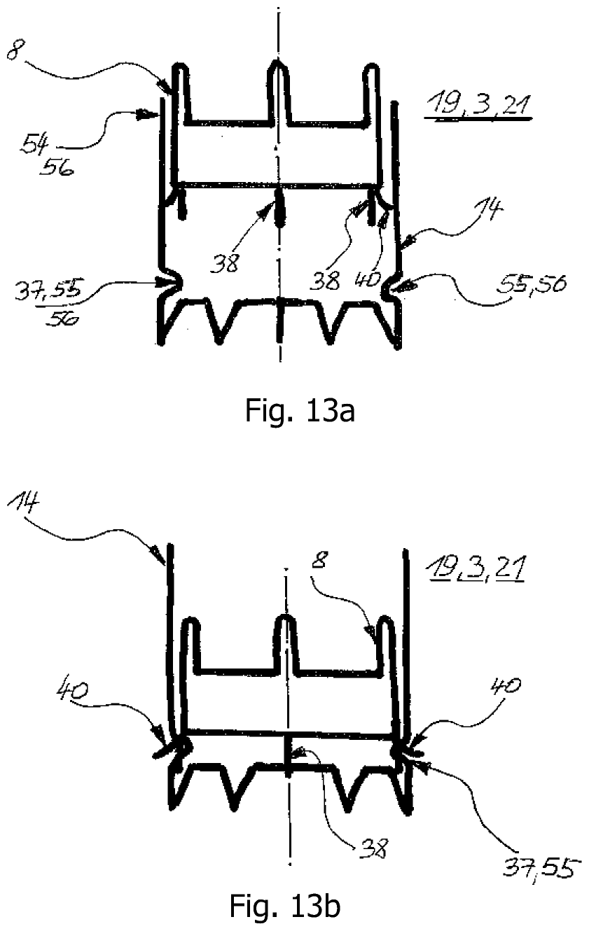

FIG. 13(a-b) shows a schematic representation of the two artificial heart valves to be joined together; the inner valve part replacement from FIG. 13a snaps into the lower outer valve part replacement as shown in FIG. 13b;

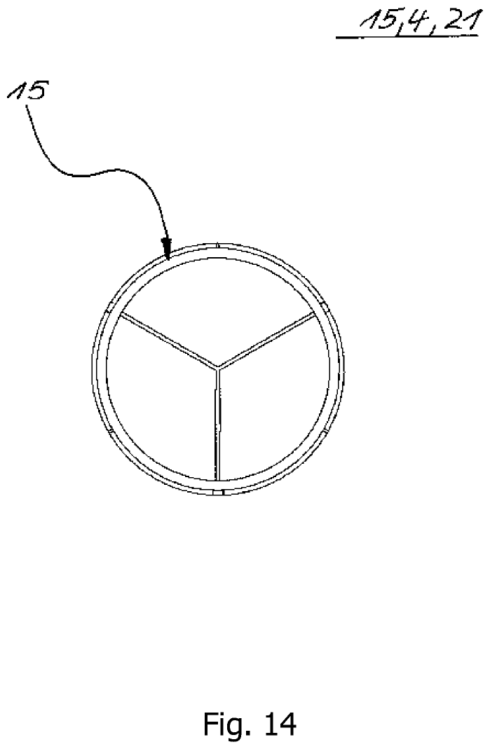

FIG. 14 shows a schematic diagram of the heart valve pockets in plan view;

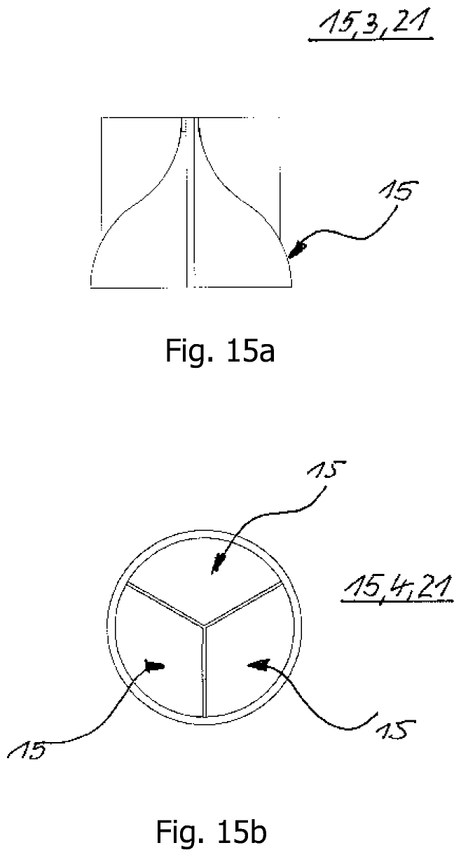

FIG. 15(a-b) shows a schematic representation of the heart valve pockets in two views;

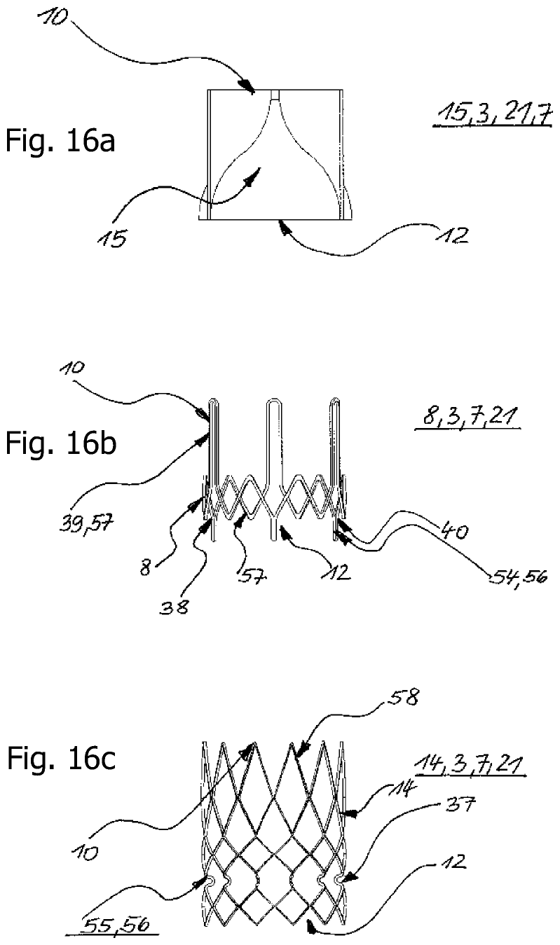

FIG. 16(a-c) shows a schematic representation of the three main parts of the two-piece artificial heart valve in three lateral views for the heart valve pockets according to FIG. 16a, the inner and outer artificial heart valve according to FIG. 16b and FIG. 16c;

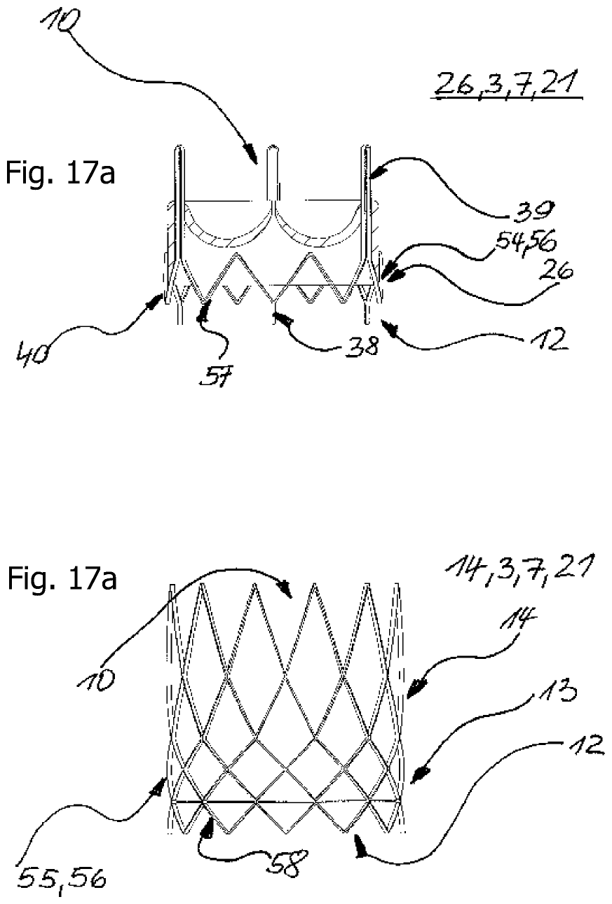

FIG. 17(a-b) shows a schematic representation of the inner artificial heart valve with the heart valve pockets FIG. 17a and the outer artificial heart valve FIG. 17b;

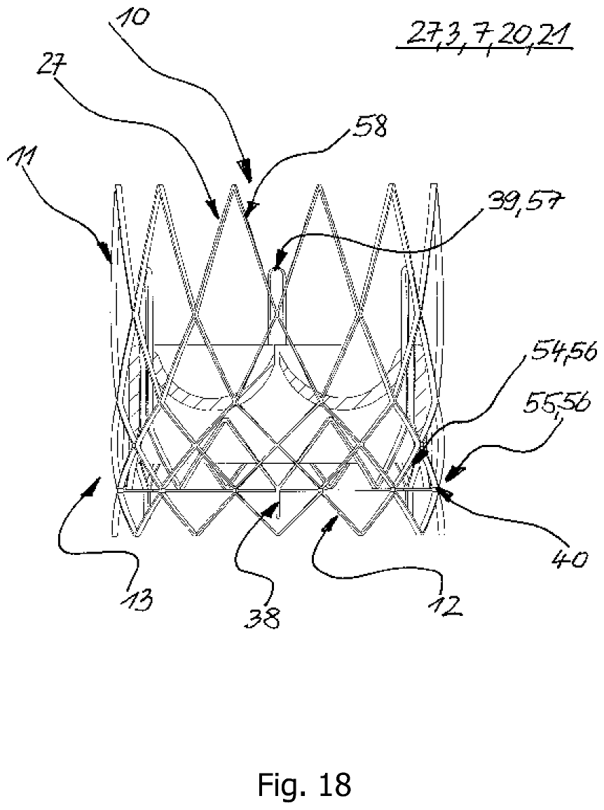

FIG. 18 shows a schematic representation of the complete two-piece artificial heart valve with heart valve pockets;

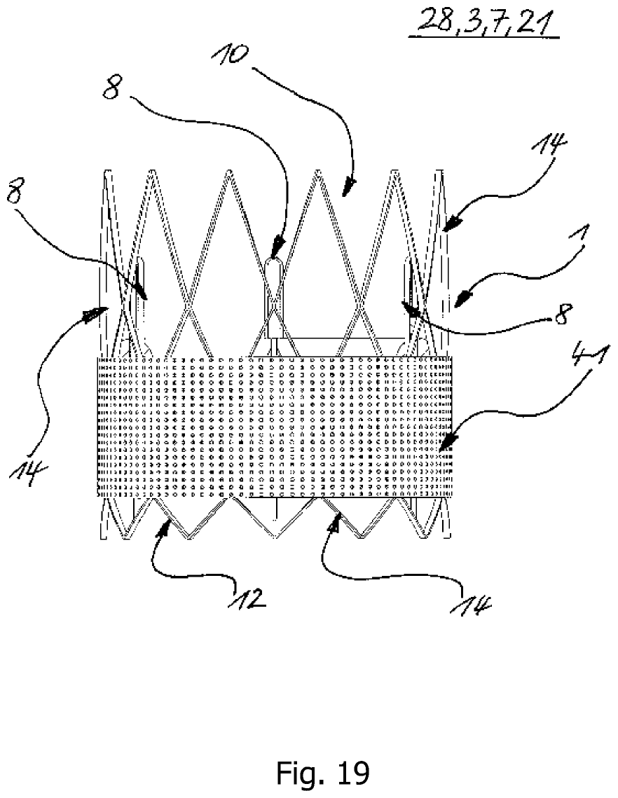

FIG. 19 shows a schematic representation of the complete two-piece artificial heart valve with heart valve pockets and, optionally, additional Dacron fleece on the outside;

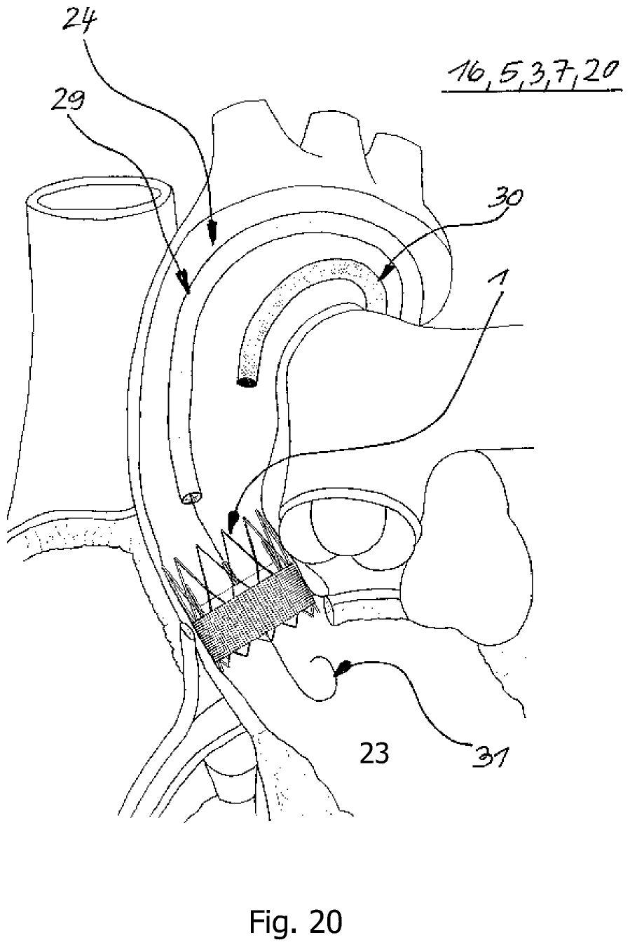

FIG. 20 shows a schematic representation of a detailed side view and partial section of the heart at the beginning of the first replacement of a new two-piece inner artificial heart valve;

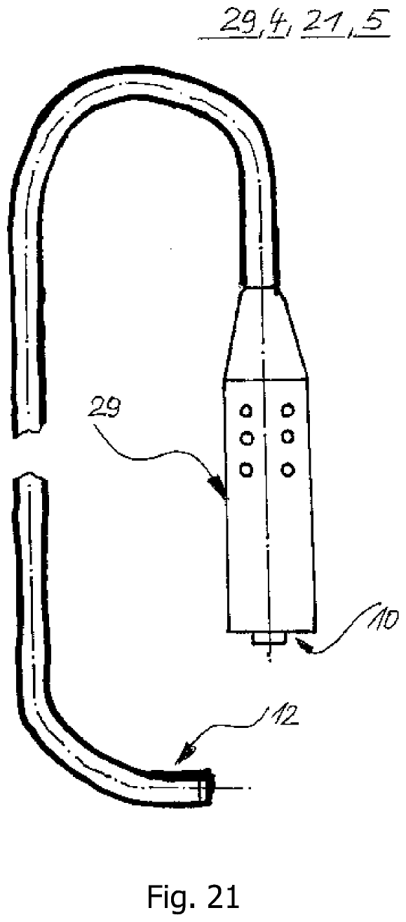

FIG. 21 shows a schematic representation of the removal tool with remote control for removing the worn inner artificial heart valve including heart valve pockets;

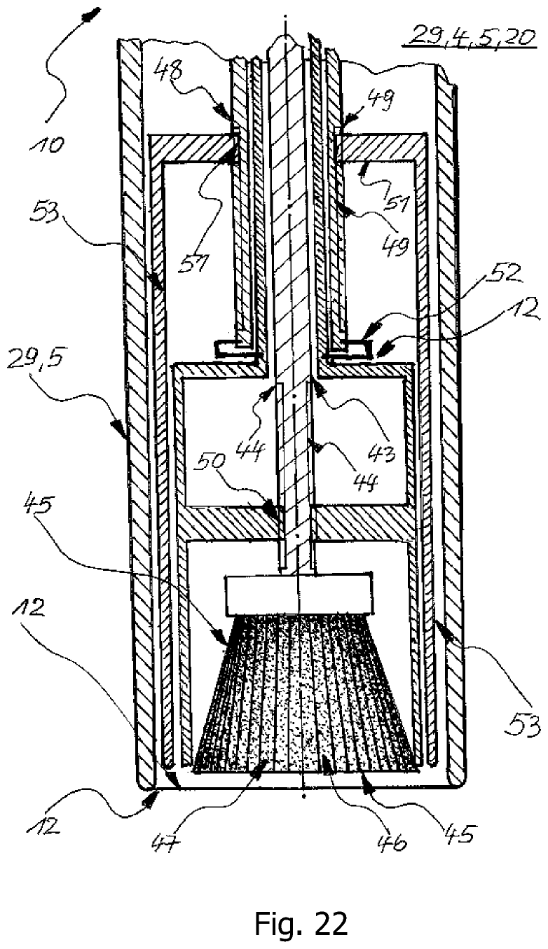

FIG. 22 shows a schematic representation with partial section of the distal end of the removal tool with remote control to explain the removal of the worn inner artificial heart valve including heart valve pockets;

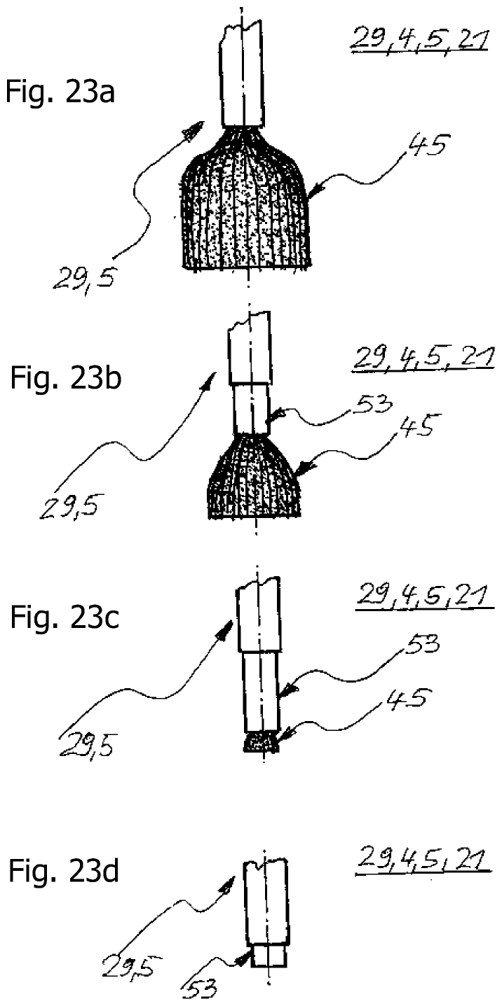

FIG. 23(a-d) shows a schematic representation of partial sequences for accommodating the worn inner artificial heart valve including heart valve pockets;

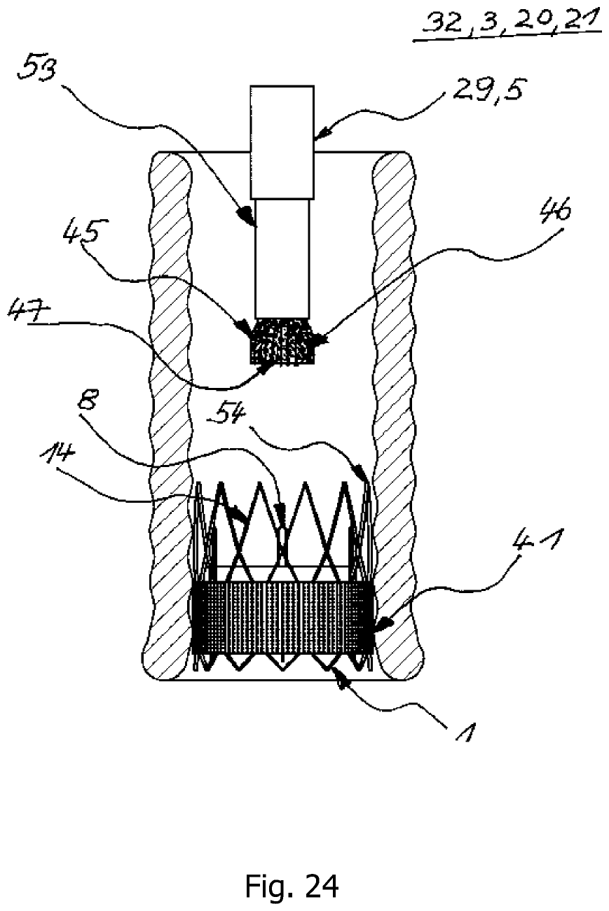

FIG. 24 shows a schematic representation of the start of the pick-up of the worn inner artificial heart valve including heart valve pockets with the removal tool with remote control;

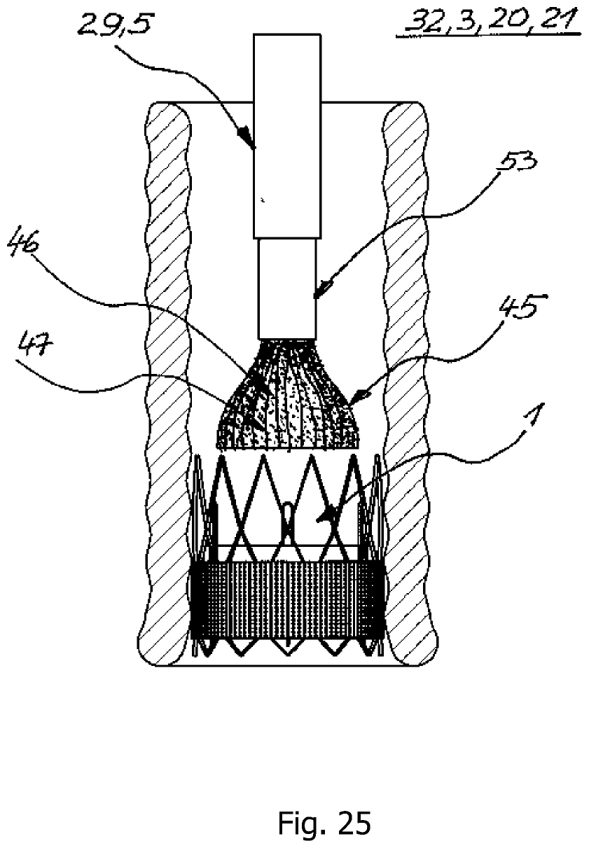

FIG. 25 shows a schematic representation of a first intermediate phase of picking up the worn inner artificial heart valve including heart valve pockets by the removal tool with remote control;

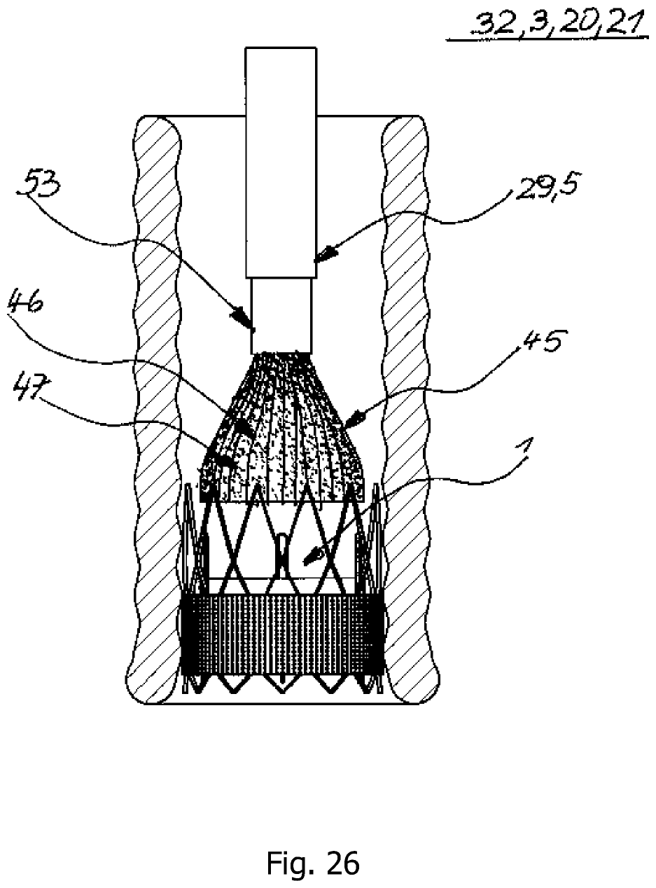

FIG. 26 shows a schematic representation of a further intermediate phase of picking up the worn inner artificial heart valve including heart valve pockets by the removal tool with remote control;

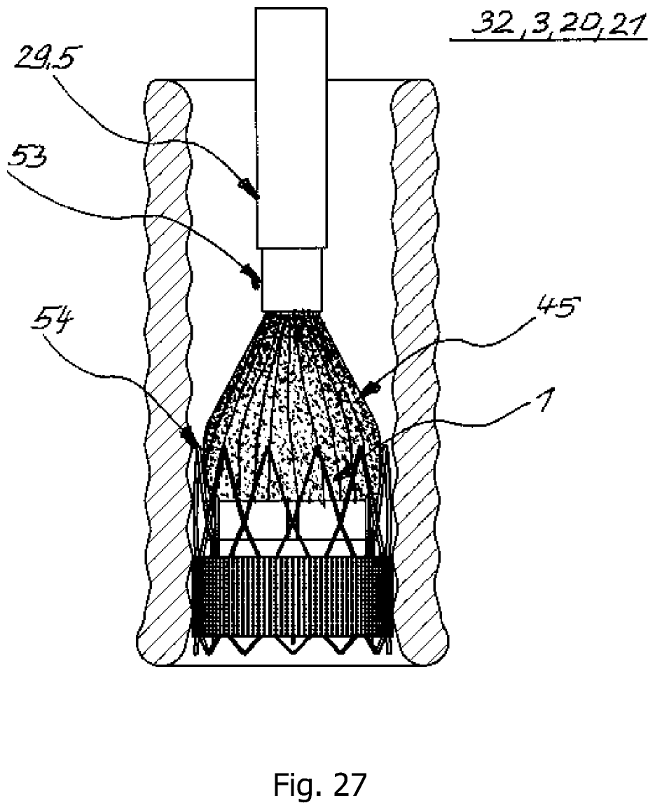

FIG. 27 shows a schematic representation of a subsequent intermediate phase of picking up the worn inner artificial heart valve including heart valve pockets by the removal tool with remote control;

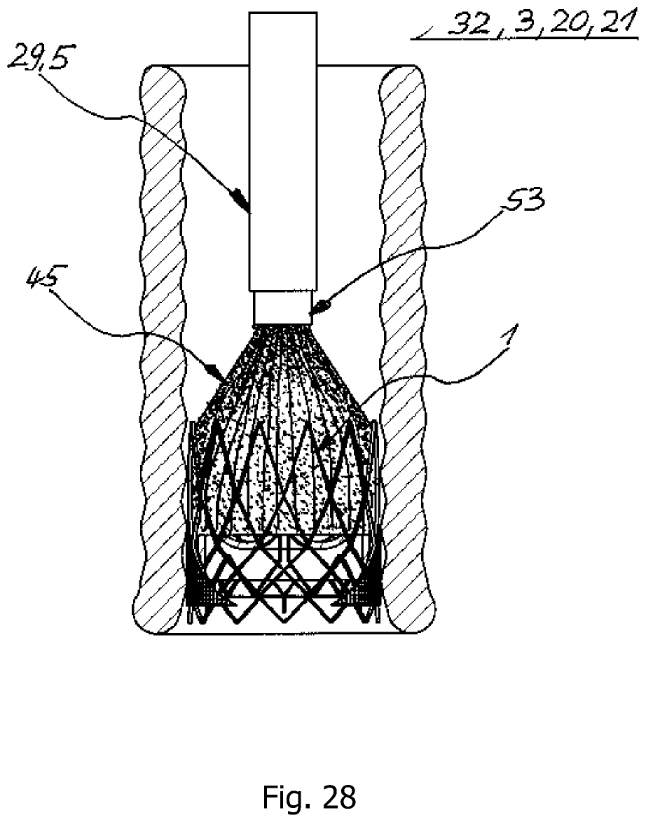

FIG. 28 shows a schematic representation of a further subsequent intermediate phase of picking up the worn inner artificial heart valve including heart valve pockets by the removal tool with remote control;

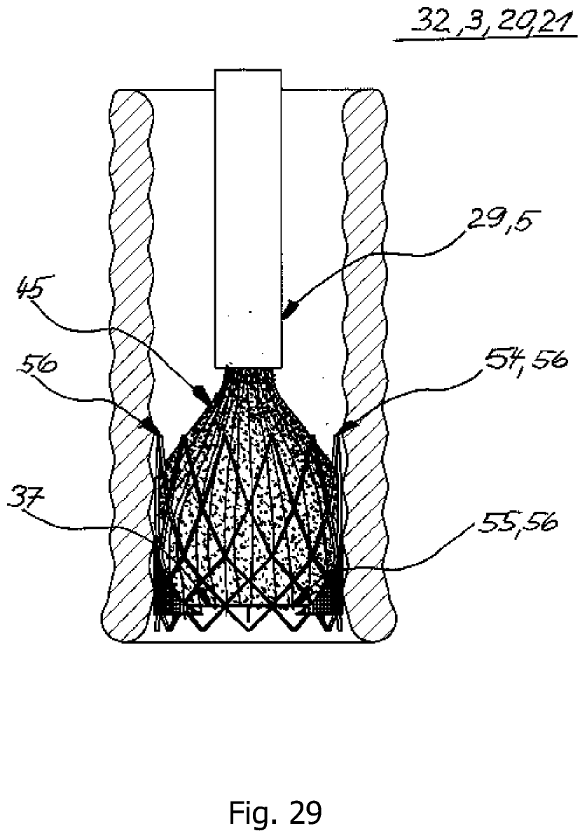

FIG. 29 shows a schematic representation of an intermediate phase of the removal of the worn inner artificial heart valve including heart valve pockets by the removal tool with remote control, which is about to be completed;

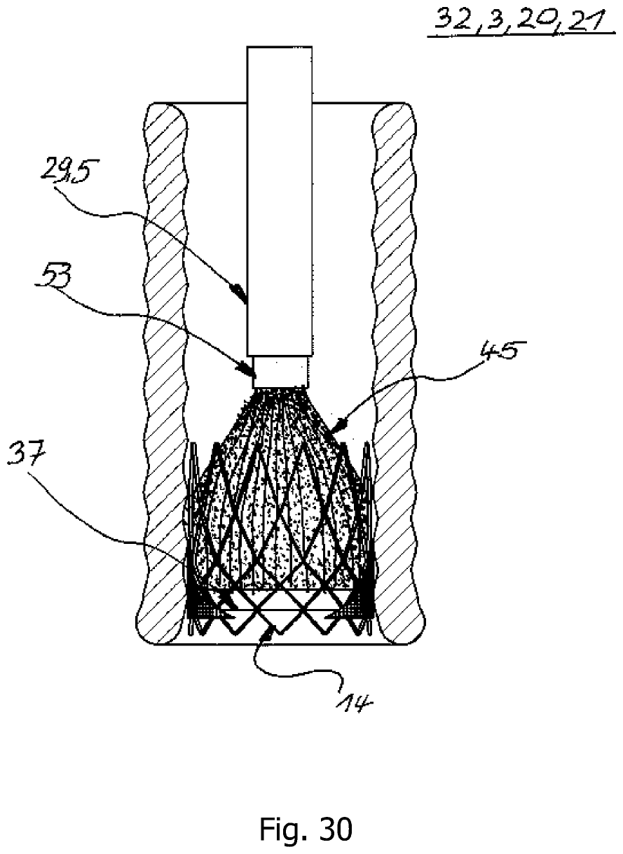

FIG. 30 shows a schematic representation of the completion of the pick-up of the worn inner artificial heart valve including heart valve pockets by the removal tool with remote control;

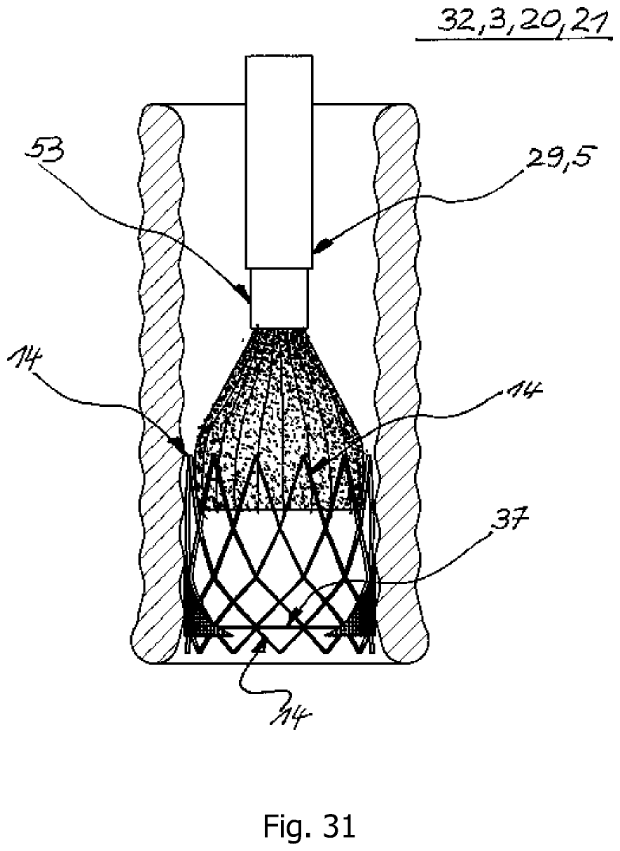

FIG. 31 shows a schematic representation of the worn inner artificial heart valve including heart valve pockets picked up by the removal tool with remote control in a first intermediate phase for later return transportation;

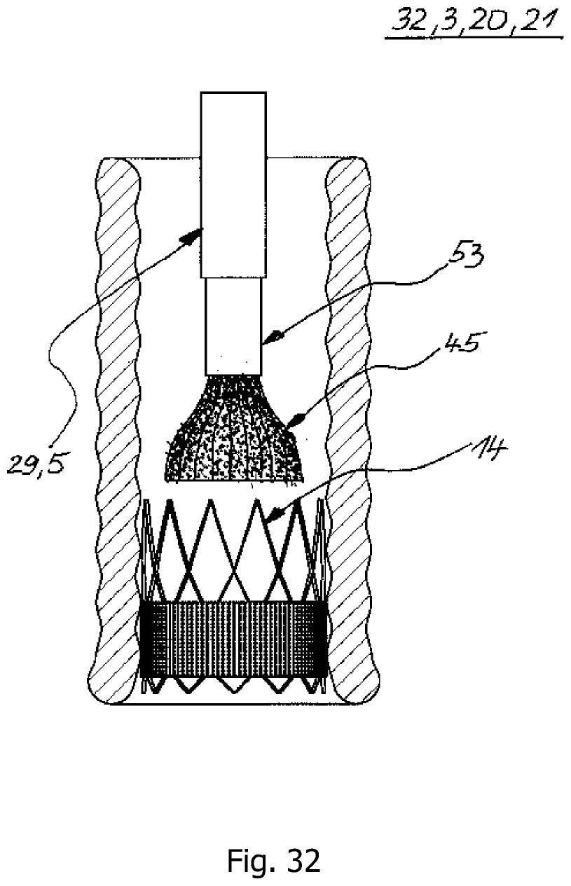

FIG. 32 shows a schematic representation of the worn inner artificial heart valve including heart valve pockets picked up by the removal tool with remote control in an advanced intermediate phase for later return transportation;

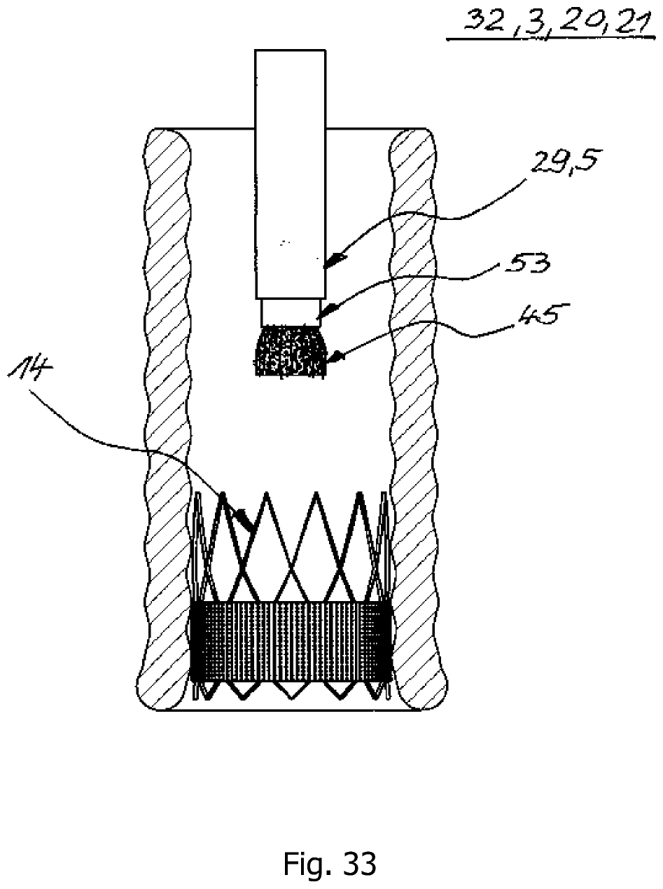

FIG. 33 shows a schematic representation of the closure of the worn inner artificial heart valve, including heart valve pockets, by the removal tool with remote control for a now possible return transportation;

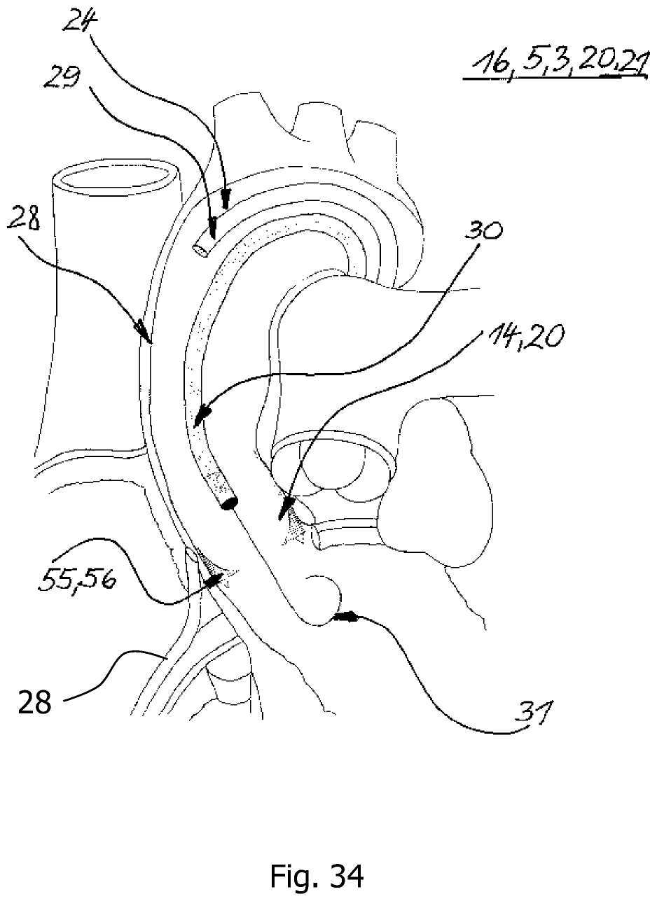

FIG. 34 shows a schematic representation of the removed worn inner artificial heart valve including heart valve pockets by the removal tool with remote control, wherein this initially remains temporarily parked in the aortic arch and representation of an implant replacement sheath for the first subsequent implantation of a new inner artificial heart valve with new heart valve pockets;

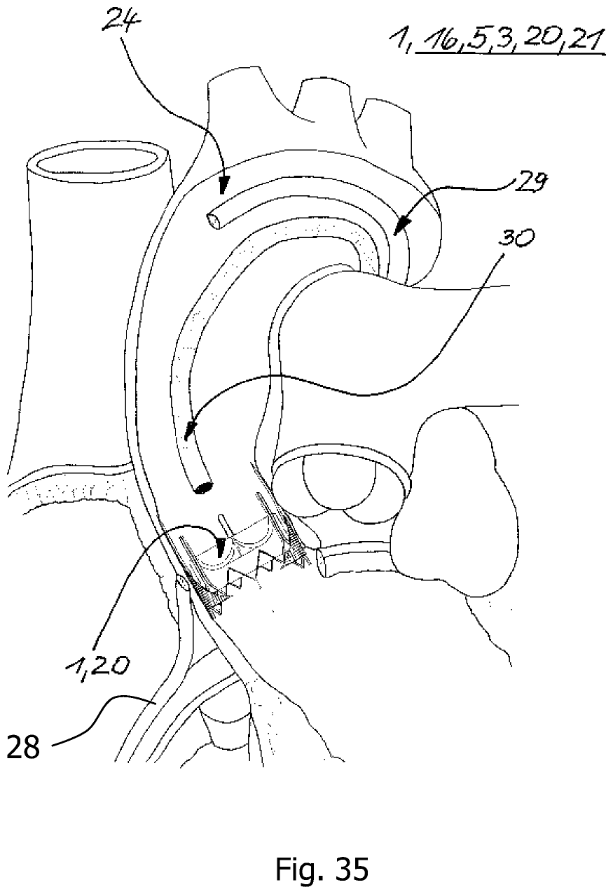

FIG. 35 shows a schematic representation of the implanted new inner artificial heart valve with new heart valve pockets analogous to the current TAVI procedure and

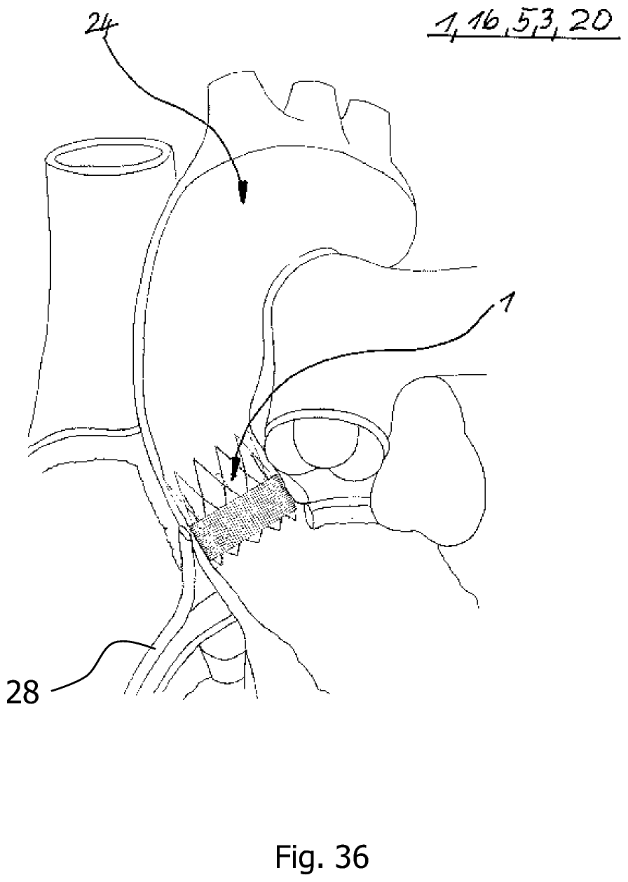

FIG. 36 shows as schematic representation of the implanted new artificial heart valve with new heart valve pockets after completion of the interventional implantation procedure.

The modular design of the interventional replaceable two-piece artificial heart valve described here as an example allows multiple interventional replacement of an inner valve module, always without ECMO, while the outer artificial heart valve remains firmly implanted and fused in the heart.

The procedure improves the life expectancy and quality of life of patients who are dependent on an artificial heart valve. The possibility of gently replacing the inner artificial heart valve, which wears out over time, significantly minimizes the risk for patients. The devices and procedures described below can be applied accordingly to all valve diseases of the heart and contribute to ultimately overcoming this disease of civilization.

All patients who are dependent on an artificial heart valve benefit from this new type of interventional artificial heart valve. These patients have the prospect of a longer life expectancy, improved performance and a better quality of life. In principle, it is possible for the lumen of the artificial heart valve to function at the same level as a native heart valve. Conventional replacement valves, on the other hand, have a smaller lumen and a lower flow, which means that they provide a poorer oxygen supply.

From the first replacement of the inner heart valve, the interventional implantation procedure used is simpler, shorter and safer for the patient than the current TAVI procedure, because the axial fixation is no longer required.

It is also possible to achieve an intervention in patients without the use of a heart-lung machine by means of such a new type of replaceable two-piece artificial heart valve and the associated procedures and devices. In addition, the use of smaller catheters and sheaths leads to gentler treatment of patients.

In order to manufacture a preferred embodiment of the replaceable two-piece artificial heart valve 1 according to FIG. 19, the corresponding metallic base bodies for the outer artificial heart valve 14 and the inner artificial heart valve 8 are first manufactured. A titanium-nickel alloy (nitinol) is used as a superelastic carrier material. Thanks to the nitinol used, the replaceable two-piece artificial heart valve can later unfold superelastically in the patient above 28 C°, which is given by the normal body temperature of approx. 36 C°.

The outer artificial heart valve 14 and the inner artificial heart valve 8 have some specific features. According to FIG. 9, the outer artificial heart valve 14 predominantly has parallelogram-shaped laser-cut closed molded elements 58. However, this is not the case with the inner artificial heart valve 8 according to FIG. 10. As can be seen in FIG. 11(a-c), the molded elements are open-open molded elements 57. This ensures that this partial area can be easily compressed when the worn inner artificial heart valve 8 is removed.

In the representation in FIG. 12, the inner artificial heart valve 8 and the outer artificial heart valve 14 are joined together. For proper locking of both components, the shoulder or inner stop 37 of the outer artificial heart valve 14 is necessary.

FIG. 13(a-b) shows the sequence of the proper joining of the outer artificial heart valve 14 and the inner artificial heart valve 8. According to FIG. 13a, the inner artificial heart valve 8 moves in the direction of the shoulder or inner stop 37 and, according to FIG. 13b, can firmly engage there via the first anchors 38 and second anchors 40. As shown in FIG. 13b, the inner and outer artificial heart valve 19 assembled as a single unit can later be implanted as a whole.

According to the representations in FIG. 14 and FIGS. 15(a-b), the corresponding 3 heart valve pockets according to FIG. 17a are attached to the segments for the attachment of a heart valve pocket 39.

FIG. 16a shows a representation of the heart valve pockets 15, FIG. 16b shows a representation of the inner artificial heart valve 8 and FIG. 16c shows a representation of the outer artificial heart valve 14.

The heart valve pockets 15 shown in FIG. 16a are inserted into the inner artificial heart valve 8 shown in FIG. 16b in an intermediate step shown in FIG. 17a. This results in the inner artificial heart valve with heart valve pockets 26, which forms the complementary component to the outer artificial heart valve 14 according to FIG. 16c.

These complementary components can now be joined together as shown in FIG. 17a and FIG. 17b. An assembly of component 26 and 14 according to FIG. 17(a-b) is thus carried out.

FIG. 18 shows the complete replaceable two-piece artificial heart valve with heart valve pockets 27 resulting from the assembly according to FIGS. 17a and 17b.

To avoid paravalvular leaks between the outer artificial heart valve 14 and aortic arch 24, it is useful at the distal end 12 of the two-piece artificial heart valve with heart valve pockets 27 to enclose this area with a suitable patch sheath 41, as shown in FIG. 19.

The interventional implantation procedure according to FIG. 1 starts with the complete replaceable two-piece artificial heart valve with heart valve pockets 27 and patch sheath 41 according to FIG. 19, which is already pretensioned in the initial implantation sheath 22.

The steps of the interventional implantation procedure are only explained using the anatomical area of the aortic arch as an example. As mentioned, the respective catheter accesses for the other heart valves differ accordingly, although the catheter application as such remains the same.

At the start of implantation, the initial implantation sheath 22 is already in the aortic arch 24, and a guide wire 31 has been placed in the center of the aortic valve 17.

According to FIG. 2, it is also possible to first position a catch net sheath 25 in the anatomical implantation area, i.e. here in the aortic arch 24, in order to catch embolizing particles in the subsequent implantation procedure. The embolizing particles can, for example, be calcifications in the area of the heart valve to be replaced, i.e. in this case the aortic valve 17, which can become detached and possibly be carried into the bloodstream. In a second step, the initial implantation sheath 22 can then be passed through the catch net sheath 25 according to FIG. 3. It is up to the surgeon to decide whether such a catch net sheath 25 is used. In the preferred exemplary embodiment according to FIG. 1, the interventional implantation procedure is performed without a catch net sheath. Such embolizations, which can lead to a stroke, can only occur in 1% of interventions.

According to FIG. 4, the initial implantation sheath 22 loaded with the replaceable two-piece artificial heart valve with heart valve pockets 27 is placed in the area of the aortic valve 17 and 36.

According to FIG. 5 to FIG. 8, the initial implantation sheath 22 loaded with the replaceable two-piece artificial heart valve with heart valve pockets 27 is now placed completely, comparable to the current TAVI procedure without ECMO, and the two-piece artificial heart valve 27 is anchored in the anatomical region, i.e. here in the region of the ejection tract of the left ventricle in the transition to the aorta, i.e. in the region of the aortic valve.

FIG. 20 shows the sequence of the first inner artificial heart valve.

The artificial heart valve is carried out by replacing the inner artificial heart valve 8, which is removed from the permanently implanted outer artificial heart valve 14 using a catheter and also replaced with a newly inserted inner artificial heart valve 8 using a catheter. In the example shown below, this is illustrated using an aortic valve replacement in the anatomical region of the aortic arch in the ejection tract of the left ventricle.

In the representation in FIG. 20, the implant exchange sheath 30 for the replacement implantation in the aortic arch 24 is already in the waiting position. The catheter tool 29 for explanting of the worn inner artificial heart valve with heart valve pockets 26 with an advanced guide wire 31, which is advanced into the area of the left ventricle 23, is also located above this in the aortic arch 24 in an advanced position. According to FIG. 21, the catheter tool 29 is operated manually or automatically at the proximal end 10 via a control unit, which is available outside the patient on the operating table.

The structure of the catheter tool 29 is shown in FIG. 22. At the distal end 12 of the catheter tool 29, there is a tool shield 45 in the form of a bell in the center. This is shaped in such a way that it protrudes completely after leaving the catheter tool 29 as shown in FIG. 27.

The bell-shape 45 of the tool shield contains porous lamellae 46 with a large number of pores 47, which allow the blood flow to pass through. A rotary movement of the first screw wire 43, which has a threaded section 44 and is guided through a threaded sleeve 50, causes tool shield in the form of the bell-shape 45 to move out of the catheter tool 29.

Accordingly, if the first screw wire 43 rotates in the opposite direction, the tool shield is returned to the bell shape 45. According to the direction of rotation of the first screw wire 43, the tool shield is completely extended into the bell shape 45 as shown in FIGS. 24 to 27.

It should be noted that when the bell-shape 45 of the tool begins to protrude, the distal end 12 of the catheter tool 29 is already below the edge of the outer artificial heart valve 14. This position is reached when the distal end 12 of the catheter tool 29 is directly at the level of the first marker 54. In the further process, the catheter tool 29 is returned in such a way that the distal end 12 of the tool shield in bell shape 45 is positioned below the first marker 54, as can be seen in FIG. 27.

According to FIG. 28 and FIG. 29, the catheter tool 29 guides the tool shield in bell shape 45 up to the shoulder or inner stop 37. The second marker 55 is also located in this position. A total of three first markers 54 are arranged axially in the same plane, each offset by 120°, in the upper region of the outer artificial heart valve 14 and a total of three second markers 55 are also arranged in the same plane, each offset by 120°, at the level of the shoulder or inner stop 37.

Due to the design of the lamellae 46 in the tool shield with the corresponding pores 47, the blood flow can take place continuously without interruption. According to FIG. 30, the inner artificial heart valve 8 with the heart valve pockets 15 is now completely separated from the outer artificial heart valve 14. From this moment on, the artificial aortic valve can no longer regulate the blood flow.

As shown in FIGS. 31 to 33, the tool shield in bell shape 45 is now returned to the catheter tool 29. This is possible because a further second screw wire 48 is present in the catheter tool 29. This second screw wire 48 has a continuous core bore in which the first screw wire 43 runs. A corresponding bearing 52 for the second screw wire 48 is provided at the distal end 12 of the second screw wire 48 so that this second screw wire 48 cannot change its position in the axial direction.

Analogous to the first screw wire 43, there is also a threaded sleeve 51 on the outer circumference of the second screw wire 48. When the second screw wire 48, which is fixed in the axial direction, is rotated, this causes the catch tube tool 53, which is connected to the threaded sleeve 51 on the second screw wire 48, to move out of the catheter tool 29. The basic shape of the catch tube tool 53 is a cylinder.

The individual FIGS. 23(a-d) show schematically how the inner artificial heart valve with heart valve pockets 26 now functions when the first screw wire 43 and the second screw wire 48 are actuated.

The first screw wire 43 moves in the opposite direction to the sequence in FIG. 24 to FIG. 30, i.e. the tool shield in bell shape 45 with the worn inner artificial heart valve with heart valve pockets 26 located therein will now attempt to retract in the direction of the catheter tool 29, as was started in FIG. 23a. The process of retracting the tool shield in bell shape 45 into the catheter tool 29 is greatly assisted by the fact that the second screw wire 48 now moves out of the catheter tool 29 in the opposite direction to the first screw wire 43, as shown in FIG. 23b and FIG. 23c. This extends the catch tube tool 53, which receives the retracting tool shield and thus the captured inner artificial heart valve and compresses it by folding it together.

According to FIG. 23d, the complete tool shield in bell shape 45 is now in the catch tube tool 53 of the catheter tool 29. In this position, the removed inner artificial heart valve is secured in the explantation tool. Immediately afterwards, the catheter tool 29 is temporarily parked in the aortic arch 24 or retracted further.

Immediately afterwards, the new inner artificial heart valve can be inserted into the existing outer artificial heart valve. This procedure is shown again in the representation from FIG. 34 using an aortic valve replacement in the anatomical area of the aortic arch and the ejection tract of the left ventricle as an example.

The implant exchange sheath 30, which is ready for implantation of the new inner artificial heart valve, is now brought into an appropriate position in order to complete the interventional implantation procedure. This position is reached when, according to FIG. 34, the distal end 12 of the implant exchange sheath 30 is above the second marker 55 and below the first marker 54, which is monitored using accompanying X-ray technology and TEE.

According to FIG. 35, the new inner artificial heart valve with heart valve pockets 26 is now implanted. It should be noted during this procedure that the two coronary arteries 28 are not closed in this example. According to FIG. 36, the catheter tool 29 and the implant exchange sheath after 2^{nd} implantation 30 have now been removed from the heart 16. Once the catheter tool 29 has been removed from the left leg artery and the implant exchange sheath 30 has been removed from the right leg artery, the interventional implantation procedure is complete.

The repeated replacement of the inner artificial heart valve with heart valve pockets 26 analogous to the TAVI procedure without the use of a heart-lung machine is only possible if the entire interventional implantation procedure is automated. To implement the automated interventional implantation procedure, corresponding microprocessors 56 are located in the area of the first markers 54 and second markers 55, which were already implanted during the initial implantation via the two-piece artificial heart valve with heart valve pockets 27 and are located in the area of the outer artificial heart valve 14 and thus remain permanently in the patient. The microprocessors are memory modules that each contain a unique identifier.

These microprocessors 56 are processors with electronic components that are miniaturized to fit into a single integrated circuit of appropriate size (approximately 0.5 to 1.5 mm) and allow systemic execution of instructions via corresponding computers located outside the patient. Spatial positions are uniquely assigned when 3 points in space are defined, which requires that 3 microprocessors 56 are placed in both the inner aortic valve replacement 8 and the outer aortic valve replacement 14. Each microprocessor is spatially assigned to a marker, as already described above for the positioning of the markers.

These processors contain position markers that can be read by a sensor system located in the approaching implantation tool. Corresponding control pulses can then be generated by a downstream control unit, which automatically enable precise axial and azimuthal positioning of the implantation tool and the inner artificial heart valve to be implanted.

With the start of the first change of the inner artificial heart valve with heart valve pockets 26 using the implant exchange sheath 30, as shown from FIG. 20, this subsection can be executed in a fully automated manner. Since the aortic valve 17 works normally up to the step shown in FIG. 29, it is important that after the phase shown in FIG. 30 (in which the aortic valve 17 is out of function), this time sequence is kept as short as possible so that support from a heart-lung machine is not required. Using appropriate control mechanisms, it can be assumed that this process will take less than 12 to 15 seconds to complete.

Suitable surface coatings can be applied to make it easier to remove the inner artificial heart valve 8 when it is worn out. This also applies to the inside of the outer artificial heart valve 14.

The coating of metallic heart implants, such as the metallic nitinol surfaces of the inner and outer artificial heart valves 8 and 14, can be used to realize various functions that lead to an improvement in the properties of the implants. In particular, diffusion barrier coatings can improve the corrosion resistance of nitinol implants and thus significantly reduce or completely prevent the release of potentially allergenic nickel ions. In this context, insulating coatings made of non-conductive materials also make it possible to shield the charge effects caused by the metal surface. The biocompatibility of metal implants can be specifically influenced by a customized coating in such a way that, on the one hand, wetting with blood, the formation and colonization with endothelial cells and the permanent bond with the metal surface are significantly promoted or improved and, on the other hand, toxic effects of the metal parts are prevented.

In contrast, coating with low-energy substrates or active substances can significantly reduce or even prevent endothelialization. With a functional coating, a controlled release of e.g. anti-inflammatory agents can also be realized over a controlled period of time. Finally, it is also possible to improve the surface properties of the heart implants in particular, such as roughness, gliding properties or scratch resistance, by means of a suitable coating.

The options for non-stick coating and drug coating of metal heart implants are described in more detail below.

Non-stick coatings can be realized on the basis of very different technologies. In biomimetic coatings, for example, micro-structured and nanostructured surfaces with hydrophobic coating substrates that have self-cleaning properties are created In a targeted manner, analogous to the lotus effect based on the example of lotus plants. This micro-structuring and nanostructuring of superhydrophobic surfaces is used technically, for example, in lotus effect façade paints. Non-stick coatings for heart implants can be realized more effectively on the basis of coatings that lead to a significantly lower surface energy of the substrate.

The surface energy is defined as the energy that must be applied to create a certain surface and is given in J/m2. For metals, the surface energy is in the range of over 40 mJ/m2, for water 72.6 mJ/m2, for polymers 39 (PVC), 30 (PE) or 18 mJ/m2 (PTFE). Coating materials with low surface energy are therefore used for non-stick coatings on metals, such as PTFE (polytetrafluoroethylene, Teflon), FEP (tetrafluoroethylene-hexafluoropropylene copolymer), PFA (copolymer of tetrafluoroethylene and perfluoroalkoxyethylene) or other fluoropolymer components that only exert a low attractive force on other substances and thus prevent the adsorption of substances such as blood components. Such fluoropolymers are quite biocompatible and have low thrombogenicity. For low-energy coating of metal surfaces of heart implants with fluoropolymers, physical and chemical processes can be used, which have already proven their worth for stents, among other things. Physical or chemical processes involve coating by physical (PVD: physical vapor deposition) or chemical (CVD: chemical vapor deposition) vapor deposition in a vacuum. In the chemical vapor deposition processes, a solid fluoropolymer layer is formed on the metal surface as a result of gaseous species reacting with the heat of the substrate.

In the plasma-assisted CVD process (PACVD), UV radiation is used to generate reactive radicals that react on the metal substrate surface even without the effect of temperature. Other processes under normal pressure are also suitable for metal coating with Teflon or other fluoropolymers. These include conventional powder coating (electrostatic process), which does not require solvents, or spray-sintering processes, wherein the Teflon coating is sprayed on and then sintered at temperatures of up to 420° C. Flawless surfaces, i.e. surfaces free of dirt and grease, are a prerequisite for the coating processes. This can be achieved using suitable pre-treatment processes, such as sandblasting or plasma etching, which can then also produce retentive adhesion patterns.

Another coating variant is the use of solvent-free fluoromonomer-containing coating lacquers that cure by polymerization. Examples of perfluorinated monomers are 2,2,3,3-tetrafluoropropyl methacrylate, 1H,1H-perfluorodecylacerylate or 1H,1H,6H,6H-perfluoro-1,6-hexanediol diacrylate, which can be used in combinations with other functionalized or crosslinking monomers. The coating layer is then cured by radical polymerization, which is initiated by heating or irradiation with UV light. Thermal polymerization is triggered by thermal initiators such as dibenzoyl peroxide or azobisisobutyronitrile, while photoinitiators such as 2,2-methoxy-2-phenylacetophenone or acyl-or bisacylphosphine oxides can be used.

To improve adhesion to the metal substrate, primers can be used that contain functionalized monomers as adhesion-promoting components, which ensure a stable chemical bond with both the metal surface and the coating material. For example, methacrylate phosphates, such as the commercially available 2-methacryloyloxyethyl dihydrogen phosphate, or chelating methacrylates, such as the commercially available 2-acetoacetoxyethyl methacrylate, can be used to coat metals with free-radically polymerizable paints.

The thrombogenicity of materials, such as plastics, in direct contact with blood remains a problem that is difficult to control. Long-term contact between blood and artificial surfaces increases the risk of thrombus formation. The reason for this is that the biomaterials behave as foreign bodies after contact with blood and activate a variety of cellular reactions in the organism and at the boundary layer of the introduced materials. This includes the activation of the body's own coagulation system, which then sets the further coagulation cascade in motion.

Various drugs can be used to inhibit coagulation in vivo. These include heparin and heparinoids, which bind to antithrombin, bringing the coagulation cascade to a standstill. Coumarins belong to the group of vitamin K antagonists and impair the synthesis of most coagulation factors. Other anticoagulant drugs include platelet. aggregation inhibitors such as aspirin (acetylsalicylic acid) or fibrinolytics. Other active substances such as antibiotics or antiseptics can also be incorporated into the coating of metal heart implants.

The drugs in question are applied to the surface of the metal heart implants as part. of a polymer coating and are then physically and/or chemically bound in or to a polymer carrier. In principle, non-water-soluble, biocompatible polymers can be used as polymeric binders. The drug and the polymer can be dissolved in a common solvent or the drug can be dispersed in a polymer solution. The polymer-drug solution or dispersion is then used to coat the metal implant using, for example, a spraying or dipping process. After drying and complete removal of the solvent—possibly in a vacuum—a ready-to-use drug-polymer coating is then available. The kinetics of drug release depend primarily on the distribution profile of the active ingredient in the polymer coating and the water absorption of the carrier polymer. Suitable carrier polymers are polycarbonates, polyester polymethacrylates, such as PMMA (polymethyl methacrylate) or biocompatible polyurethanes. In principle, fluoropolymers are also suitable, but these only have limited solubility and minimal water absorption.

Polymer coatings based on solvent-free radical polymerizable resins, which contain the drug dissolved or dispersed, allow a more targeted release of the active ingredient. Radical polymerization of e.g. methacrylate resins produces insoluble, cross-linked polymers in which the drug is molecularly dissolved and/or dispersed. The addition of hydrophilic monomers, such as 2-hydroxyethyl methacrylate, can increase water absorption and thus accelerate the release of the active ingredient. In contrast, hydrophobic comonomers, such as benzyl methacrylate, lead to a reduction in water absorption and thus to a delayed release of the active ingredient.

Furthermore, the kinetics of the active ingredient release can be controlled by the degree of crosslinking of the polymers. Thus, the release of the active ingredient can be significantly delayed with increasing content of crosslinking monomers, e.g. dimethacrylates, in the resin mixture and with increasing functionality of the methacrylates, e.g. tri- and tetramethacrylates. In this context, polymer-bound active ingredients can also be used, i.e. polymerizable resins that contain polymerizable active ingredient derivatives, such as a methacrylated aspirin, alone or in addition to the already dissolved or dispersed active ingredient. The rate of drug release also depends on the rate of hydrolysis of the drug-monomer or drug-polymer bond. Finally, the above-mentioned perfluorinated monomers can also be used for drug coatings to realize drug coatings with low surface energy.

Another variant for a drug coating is to create a more or less porous implant surface for the storage of the drug or drugs before coating. This can also extend the release period. Gradient coatings with decreasing drug concentration from the inside to the outside can also be realized. Such gradient coatings can be easily produced by multiple coating with polymer-drug combinations of different drug concentrations.

The drug coatings described above are of the diffusion type, wherein the active ingredient diffuses from the polymer matrix into the environment (bloodstream). Drug coatings of the so-called erosion type can be produced with biodegradable polymers, such as poly(glycolide), poly(lactide) or polyanhydrides. The drug or a derivative thereof is dissolved or dispersed in the biodegradable polymer and the metal heart implant is coated with the polymer-drug combination. The active ingredient is then released by hydrolytic and/or enzymatic degradation of the carrier polymers and thus also by gradual dissolution of the coating. Such erosion-type active ingredient-polymer combinations can also be produced on the basis of polymerizable resins. Suitable hydrolytically or biodegradable monomer components can be, for example, methacrylate-functionalized lactic acid or glycolic acid oligomers or other oligomeric esters, anhydrides or amino acids.

Finally, it is possible to use a combination of diffusion-and erosion-type coatings, with the outer layer being formed by the erosion-type polymer-agent combination.

After applying the appropriate surface coatings, the corresponding heart valve pockets 15 are incorporated into the inner artificial heart valve 8.

The aspects of the invention have been explained with reference to exemplary embodiments. Further embodiments are shown in the subclaims. Further modifications are also possible within the scope of skill in the art.

### List of Reference Signs

1 Two-piece artificial heart valve with heart valve pockets and patch sheath
2 Front view
3 Side view
4 Top view
5 Detail view
6 Sectional view
7 Spatial representation
8 Inner artificial heart valve
9 Lasered sheath
10 Proximal end
11 Proximal retention area
12 Distal end
13 Distal retention area
14 Outer artificial heart valve
15 Heart valve pockets
16 Heart
17 Aortic valve
18 Molded elements
19 Inner and outer artificial heart valve
20 Partial section
21 Schematic representation
22 Initial implantation sheath
23 Left ventricle
24 Aortic arch
25 Catch net sheath
26 Inner artificial heart valve with heart valve pockets
27 Two-piece artificial heart valve with heart valve pockets
28 Coronary artery
29 Catheter tool
30 Implant exchange sheath
31 Guide wire
32 Partial view of the aortic arch
33 Inferior vena cava
34 Superior vena cava
35 Right ventricle
36 Aortic valve lateral section
37 Shoulder or inner stop
38 First anchor
39 Segment for attaching a heart valve pocket
40 Second anchor
41 Patch sheath
42 Gap between inner and outer artificial heart valve
43 First screw wire
44 Threaded section to 43
45 Tool shield in bell shape
46 Lamella
47 Pores
48 Second screw wire
49 Threaded section to 48
50 Threaded sleeve to 43
51 Threaded sleeve to 48
52 Bearing for 48
53 Catch tube tool
54 First marker
55 Second marker
56 Microprocessor
57 Open molded element
58 Closed molded element

1. Artificial heart valve (27) having a two-piece structure, consisting of an outer artificial heart valve (14) with means for permanently anchoring the artificial heart valve at the implantation site and an inner artificial heart valve (8) with artificial heart valve pockets (15) which can be replaced independently of the outer artificial heart valve (14) anchored at the implantation site and inserted into the outer artificial heart valve (14), wherein the outer artificial heart valve has formations for a defined positioning of the inner artificial heart valve (8) and the inner artificial heart valve (8) has formations for anchoring the inner artificial heart valve (8) in the outer artificial heart valve (14).

2. Artificial heart valve according to claim 1,
characterized in that
the outer artificial heart valve (14) is an arrangement of closed molded elements (58) cut from a tube in the form of a net, wherein the formations for the defined contact are a series of shoulders (37) projecting into the interior of the arrangement, and
the inner artificial heart valve (8) has a shape-variably compressible wire-like meandering structure of open mold elements (57), wherein the formations for anchoring are formed as a series of first anchors (38) and second anchors (40) and the inner artificial heart valve has a series of segments (39) for fastening the artificial heart valve pockets (15).

3. Artificial heart valve according to claim 1,
characterized in that
the outer artificial heart valve (14) has a patch sheath (41) surrounding its distal end (12) for sealing paravalvular leaks.

4. Artificial heart valve according to claim 1,
characterized in that
the inner artificial heart valve (8) and the outer artificial heart valve (14) consist of a rotationally symmetrical, elastic nitinol sheath (9), wherein the open molded elements (57) and the closed molded elements (58) are cut out of the nitinol sheath (9).

5. Artificial heart valve according to claim 1,
characterized in that
the outer artificial heart valve (14) in the region of the projecting shoulders (37) and the inner artificial heart valve (8) in the region of the first anchors (38) and/or the second anchors (40) each have sections forming X-ray contrasts as markers (55, 54).

6. Artificial heart valve according to claim 5,
characterized in that
a microprocessor (56) is arranged in the region of each of the markers (55, 54).

7. Artificial heart valve according to claim 1,
characterized in that
the inner artificial heart valve (8) and/or the outer artificial heart valve (14) has a non-stick coating and/or a pharmacologically effective coating.

8. Device for performing catheter-based implantation of an artificial heart valve (27), consisting of
a tube-like flexible initial implantation sheath (22), a guide wire (31) movable within the initial implantation sheath and the two-piece artificial heart valve (27), which is displaceable within the initial implantation sheath (22) along the guide wire (31) and consists of an outer artificial heart valve (14) and an inner artificial heart valve (8) or a displaceable inner heart valve insert (8) for insertion into an already pre-implanted outer heart valve insert (14).

9. Device according to claim 8,
characterized by
a tubular-flexible catch net sheath (25) supplementing the initial implantation sheath (22) and having a particle catch net that can be unfolded at the distal end and covers the valve cross-section, wherein the initial implantation sheath can be passed through the particle catch net from the outside to the intended implantation site.

10. Device for an in-situ replacement of an inner artificial heart valve (8) with a new inner artificial heart valve (8) in an implanted two-piece artificial heart valve (27), comprising the inner artificial heart valve (8) and an outer artificial heart valve (14),
consisting of
an explantation catheter for an explantation of the inner artificial heart valve (8), comprising a tubular-flexible explantation sheath (29) with a guide wire (31) arranged in the explantation catheter and an explantation tool guided in the explantation sheath with means for gripping and releasing anchors of the inner artificial heart valve (8) with a proximal operating unit (10) for semi-automatic operation of the explantation tool and an implantation catheter, comprising a tubular-flexible implant exchange sheath (30) with means for feeding and inserting a new inner artificial heart valve (8) into the outer artificial heart valve (14).

11. Device according to claim 10,
characterized in that
the explantation tool has the following structure:
a first feed means having a bell-shaped tool shield (45) attached to the distal end of the first feed means (43) and a guide means (50) accommodating the tool shield for guiding the first feed means,
a second feed means with a continuous recess accommodating the first feed means,
a bearing (52) connecting the second feed means (48) to the guide means (50) for the tool shield and fixing the second feed means (48) in the axial direction,
a catch tube tool (53) for the tool shield (45) which can be moved by the second feed means.

12. Device according to claim 11,
characterized in that
the first feed means is a first inner screw wire (43) with a tool shield (45) attached to the distal end of the first screw wire (43) and a tool shield threaded sleeve (50) accommodating the tool shield for guiding the first screw wire (43).

13. Device according to claim 11,
characterized in that
the second feed means is a second outer screw wire (48) with a continuous core bore accommodating the inner screw wire (43) and an outer threaded section (49) with a bearing (52) connecting the second outer screw wire (48) to the threaded sleeve (50) and fixing the outer screw wire (48) in the axial direction, wherein a catch tube threaded sleeve (51) guiding the outer threaded section (49) of the outer screw wire (48) and a catch tube tool (53) connected to the catch tube threaded sleeve (51) and concentrically surrounding the tool shield threaded sleeve (50) are provided.

14. Device according to claim 11,
characterized in that,
in the unfolded state, the tool shield (45) completely peripherally covers the inner artificial heart valve (8) located in the outer artificial heart valve (14) up to the shoulders (37) located in the outer artificial heart valve (14) and engages in the peripheral space between the inner artificial heart valve (8) and the outer artificial heart valve (14), wherein anchoring formations of the inner artificial heart valve (8) are released from the outer artificial heart valve (14) by this intervention and instead engage in perforations of the tool shield (45).

15. Method for explanting an artificial heart valve comprising the following method steps of:
using a two-piece artificial heart valve implanted at the implantation site with an outer artificial heart valve (14) firmly implanted at the implantation site and an inner artificial heart valve (8) detachably anchored in the outer artificial heart valve (14),
advancing an explantation catheter with a tubular-flexible explantation sheath (29) and a gripping and removal tool contained in the explantation sheath into the region of the implantation site,
extending the gripping and removal tool from the explantation sheath (29) onto the inner artificial heart valve (8),
gripping the inner artificial heart valve (8) and releasing the anchoring with the outer artificial heart valve (14) by the gripping and removal tool,
withdrawing the gripped inner artificial heart valve (8) by the gripping and removal tool and retrieval of the inner artificial heart valve (8) in the explantation sheath (29) of the explantation catheter,
withdrawing the explantation catheter with the inner artificial heart valve (8) retrieved in the explantation sheath (29).

16. Method according to claim 15,
characterized in that
the gripping and removal tool is extended from the explantation sheath (29) and the inner artificial heart valve (8) is gripped by extending and deploying a tool shield (46), wherein the tool shield (46) is lowered over the inner artificial heart valve (8), thereby releasing the anchoring of the inner artificial heart valve (8) to the outer heart valve projection (14) and hooking the inner heart valve projection (8) onto the tool shield (46).

17. Method according to claim 15,
characterized in that
the retraction of the captured inner artificial heart valve (8) by the gripping and removal tool and the retrieval of the captured inner artificial heart valve (8) are carried out with the following method steps of:
retracting the tool shield (45) with the hooked inner artificial heart valve (8) into a catch tube tool (53) emerging from the explantation sheath (29) and compressing the inner artificial heart valve (8) in the catch tube tool (53),
retracting the catch tube tool (53) with the compressed inner artificial heart valve (8) contained therein into the explantation sheath (29).

18. Method for reimplanting an artificial heart valve with the following steps of:
using a two-piece artificial heart valve (27), consisting of an inner artificial heart valve (8) and an outer artificial heart valve (14), wherein the outer artificial heart valve (14) is firmly implanted in the anatomical region of the heart valve and remains permanently at the implantation site and the inner artificial heart valve (8) is advanced into the region of the pre-implanted outer artificial heart valve (14) by means of an implantation catheter and is inserted into the outer artificial heart valve (14) by means of an implantation tool located in the implantation catheter and is anchored via anchoring means located on the inner artificial heart valve (8).

19. Method according to claim 15,
characterized in that
the explantation and reimplantation of the artificial heart valve is carried out as a directly successive explantation and reimplantation, wherein, in a first step, a first inner artificial heart valve (8) is explanted from the outer artificial heart valve (14) via the explantation catheter and the explantation sheath (29) and, in an immediately subsequent second step, a second inner artificial heart valve (8) is inserted into the outer artificial heart valve (14) via the implantation catheter.

20. Method according to claim 15,
characterized in that
the individual steps during explantation and/or reimplantation are carried out automatically by means of an automatically operated catheter tool arranged at the proximal end of the explantation catheter and/or the implantation catheter.
